(19) United States
(12) Patent Application Publication
(10) Pub. No.: US 2026/0103447 A1
(43) Pub. Date: Apr. 16, 2026

(54) METHODS FOR PREPARING PSMA CONJUGATES

(71) Applicant: Endocyte, Inc., East Hanover, NJ (US)

(72) Inventors: Iontcho R. VLAHOV, West Lafayette, IN (US); Hari Krishna R. SANTHAPURAM, West Lafayette, IN (US); Albert E. FELTEN, West Lafayette, IN (US); Christopher P. LEAMON, West Lafayette, IN (US); Daniel S. RENO, West Lafayette, IN (US); Katheryn M. STANFORD, West Lafayette, IN (US)

(21) Appl. No.: 19/374,943

(22) Filed: Oct. 30, 2025

Related U.S. Application Data
(63) Continuation of application No. 17/612,283, now Pat. No. 12,473,265.
(60) Provisional application No. 62/850,119, filed on May 20, 2019.
(60) Provisional application No. 62/910,777, filed on Oct. 4, 2019.
(60) Provisional application No. 62/912,353, filed on Oct. 8, 2019.

(51) Int. Cl. C07D 257/02

(52) U.S. Cl. CPC C07D 257/02

(57) ABSTRACT

This disclosure relates to processes for preparing compounds that are useful in the treatment of disease, such as cancer, in mammals. In particular, the invention described herein pertains to processes for preparing compounds capable of targeting PSMA expressing cells and useful in the treatment of diseases caused by PSMA expressing cells, such as prostate cancer.

### CROSS-REFERENCE TO RELATED APPLICATIONS

This application is a continuation of U.S. patent application Ser. No. 17/612,283, filed Nov. 18, 2021, which is a national stage application of International Patent Application No. PCT/US2020/033584, filed May 19, 2020, which claims the benefit of, and priority to, U.S. Provisional Patent Application No. 62/850,119, filed May 20, 2019, U.S. Provisional Patent Application No. 62/910,777, filed Oct. 4, 2019, and U.S. Provisional Patent Application No. 62/912,353, filed Oct. 8, 2019, the entire disclosures of each of which is incorporated herein by reference.

### TECHNICAL FIELD

This disclosure relates to processes for preparing compounds that are useful in the treatment of disease, such as cancer, in mammals. In particular, the invention described herein pertains to processes for preparing compounds capable of targeting PSMA expressing cells and useful in the treatment of diseases caused by PSMA expressing cells, such as prostate cancer.

### BACKGROUND

The prostate is a male reproductive organ and functions to produce and store seminal fluid that provides nutrients and fluids for the survival of sperm introduced into the vagina during reproduction. Like other tissues, the prostate gland may develop either malignant (cancerous) or benign (non-cancerous) tumors. In fact, prostate cancer is one of the most common male cancers in western societies, and is the second leading form of malignancy among American men. Current treatment methods for prostate cancer include hormonal therapy, radiation therapy, surgery, chemotherapy, photodynamic therapy, and combination therapy. However, many of these treatments affect the quality of life of the patient, especially for those men who are diagnosed with prostate cancer over age 50. For example, the use of hormonal drugs is often accompanied by side effects such as osteoporosis and liver damage. Such side effects might be mitigated by the use of treatments that are more selective or specific to the tissue being responsible for the disease state, and avoid non-target tissues like the bones or the liver.

Prostate-specific membrane antigen (PSMA) is a biomarker that is overexpressed on prostate cancer. PSMA is over-expressed in the malignant prostate tissues when compared to other organs in the human body such as kidney, proximal small intestine, and salivary glands. PSMA is also expressed on the neovasculature within many non-prostate solid tumors, including lung, colon, breast, renal, liver and pancreatic carcinomas, but not on normal vasculature. PSMA is also expressed minimally in brain. PSMA is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. For example, PSMA plays a role in the central nervous system, where it metabolizes N-acetyl-aspartyl glutamate (NAAG) into glutamic and N-acetyl aspartic acid. PSMA also plays a role in the proximal small intestine where it removes γ-linked glutamate from poly-γ-glutamated folate and α-linked glutamate from peptides and small molecules. However, PSMA's particular function on prostate cancer cells remains unresolved.

Unlike many other membrane-bound proteins, PSMA undergoes rapid internalization into the cell in a similar fashion to cell surface bound receptors like vitamin receptors. PSMA is internalized through clathrin-coated pits and subsequently can either recycle to the cell surface or go to lysosomes. Accordingly, diagnostic, imaging, and therapeutic agents can be targeted to PSMA for delivery into PSMA expressing cells, such as prostate cancer cells.

Described herein are processes for preparing compounds capable of binding to PSMA. Also described herein are processes for preparing compounds capable of targeting PSMA for delivery of diagnostic, imaging, and therapeutic agents.

It has been unexpectedly discovered that the conjugates prepared by the processes described herein exhibit high affinity for PSMA. It has also been discovered that the compounds prepared by the processes described herein are efficacious in treating diseases caused by pathogenic cells that express PSMA, such a prostate cancer cells.

It has been disclosed that certain conjugates exhibit high affinity for PSMA. It has also been disclosed that the conjugates are efficacious in treating diseases caused by pathogenic cells that express PSMA, such a prostate cancer cells. One such conjugate is

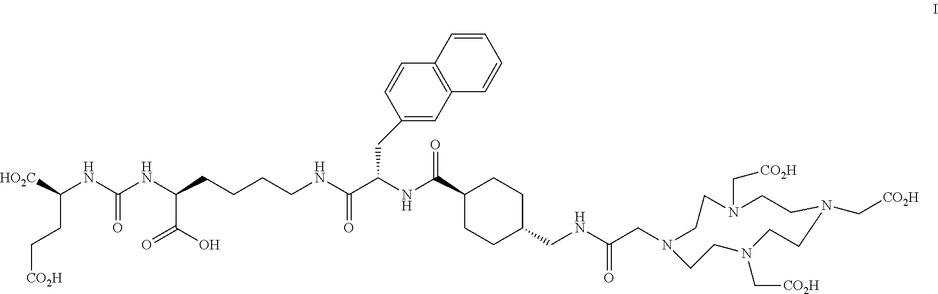

(a.k.a. PSMA-617) described in WO2015/055318 A1.

Because of the demand for pharmaceutical products, such as PSMA-617, there exists a need to provide synthetic methods capable of delivering large quantities of such products at a lower cost and in high purity. Described herein are processes for preparing compounds capable of binding to PSMA, such as PSMA-617.

### SUMMARY

In one aspect, the present disclosure provides a process for preparing a compound that is useful for the treatment of disease, especially cancer, in a mammal, including a human.

In one embodiment, the present disclosure provides a process for preparing a compound for treating abnormal cell growth in a mammal, including a human, in need of such treatment.

In another embodiment, the abnormal cell growth is cancer. In another embodiment, the cancer is prostate cancer, metastatic prostate cancer, and metastatic castration resistant prostate cancer

In another aspect, the disclosure provides processes for preparing a compound of the formula I

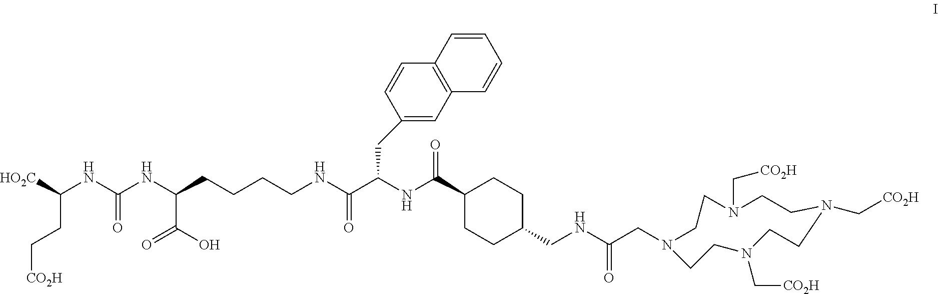

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A process for preparing a compound of the formula I

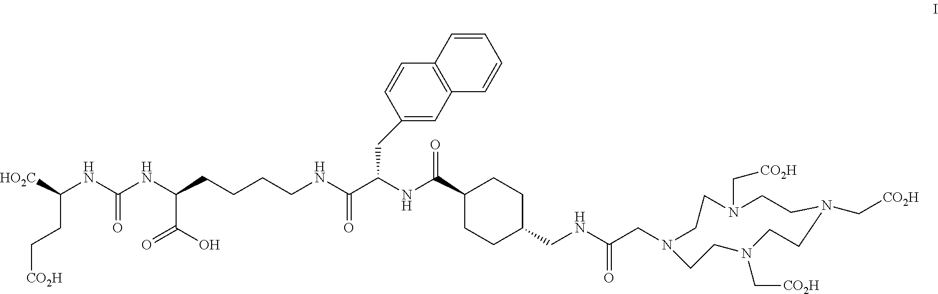

comprising

a. contacting a resin-based compound of the formula A

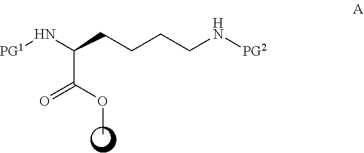

with a first deprotecting agent in a polar aprotic solvent to provide a resin-based compound of the formula A′

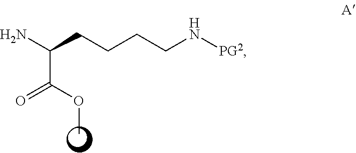

or

b. contacting the resin-based compound of the formula A′ with a compound of the formula B

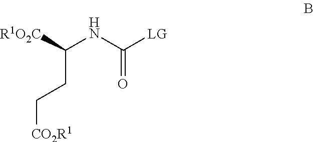

in the presence of an organic solvent and a base to provide a resin-based compound of the formula C

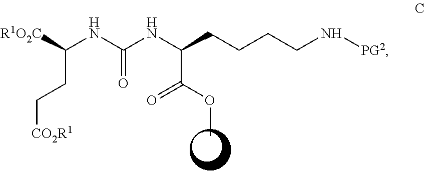

or

c. contacting the resin-based compound of the formula C with a second deprotecting agent in the presence of an organic solvent to provide a resin-based compound of the formula C′

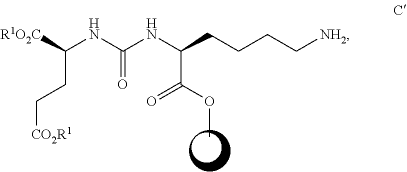

or

d. contacting the resin-based compound of the formula C′ with PG¹-3-(2-napthyl)-L-alanine in the presence of a polar aprotic solvent, a coupling agent, and a base to provide a resin-based compound of the formula D

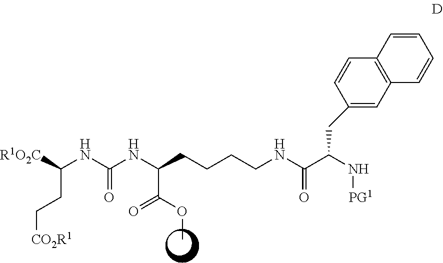

or

e. contacting the resin-based compound of the formula D with the first deprotecting agent in a polar aprotic solvent to provide a resin-based compound of the formula D′

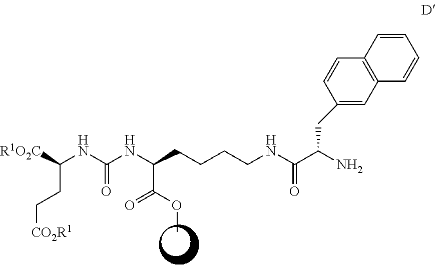

or

f. contacting the resin-based compound of the formula D′ with PG¹-tranexamic acid in the presence of a polar aprotic solvent, a coupling agent, and a base to provide a resin-based compound of the formula E

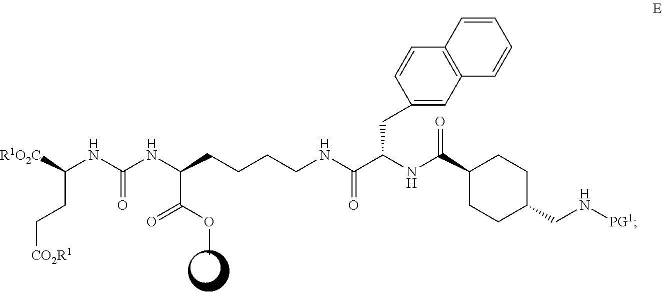

or

g. contacting the resin-based compound of the formula E with the first deprotecting agent in a polar aprotic solvent to provide a resin-based compound of the formula E′

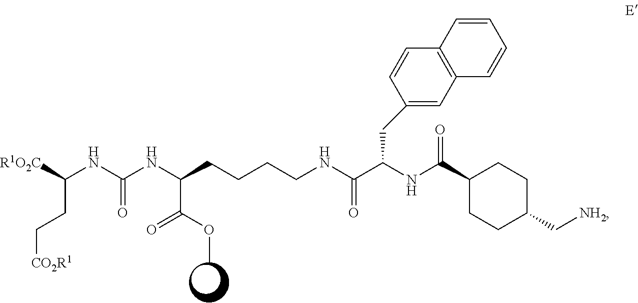

or

h. contacting the resin-based compound of the formula E′ with a compound of the formula E^{2′}

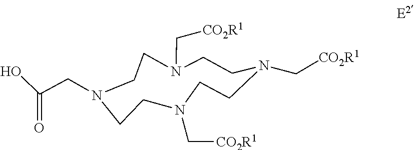

in the presence of a polar aprotic solvent, a coupling agent, and a base to provide a resin-based compound of the formula F

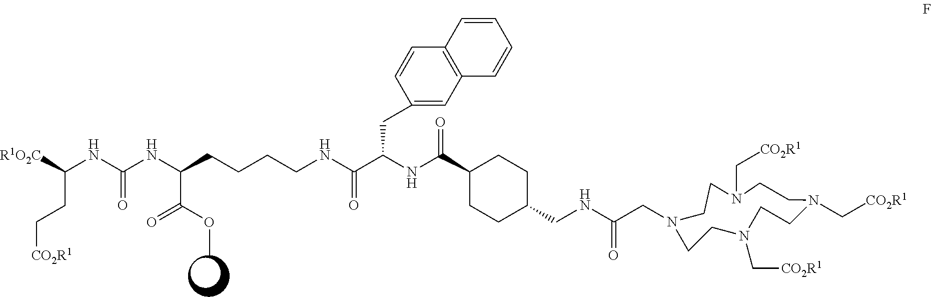

or

i. contacting the resin-based compound of the formula F with a third deprotecting agent and/or a cleavage reagent in an organic solvent,

wherein PG¹ and PG² are amine protecting groups, LG is a leaving group, and R¹ is a C₁-C₄ alkyl or a cyclohexyl.

2. The process of clause 1, wherein the first deprotecting agent of step (a) is selected from the group consisting of piperidine, morpholine, 1,8-Diazabicyclo[5.4.0]undec-7-ene (DBU), diethylamine (DEA), dicyclohexamine, 4-methylpiperidine (4MP), Tris(2-aminoethyl)amine, pyridine and collidine.

3. The process of any one of the preceding clauses, wherein the first deprotecting agent of step (e) is selected from the group consisting of piperidine, morpholine, 1,8-Diazabicyclo[5.4.0]undec-7-ene (DBU), diethylamine (DEA), dicyclohexamine, 4-methylpiperidine (4MP), Tris(2-aminoethyl)amine, pyridine and collidine.

4. The process of any one of the preceding clauses, wherein the first deprotecting agent of step (g) is selected from the group consisting of piperidine, morpholine, 1,8-Diazabicyclo[5.4.0]undec-7-ene (DBU), diethylamine (DEA), dicyclohexamine, 4-methylpiperidine (4MP), Tris(2-aminoethyl)amine, pyridine and collidine.

5. The process of any one of the preceding clauses, wherein the first deprotecting agent of step (a) is piperidine.

6. The process of any one of the preceding clauses, wherein the first deprotecting agent of step (e) is piperidine.

7. The process of any one of the preceding clauses, wherein the first deprotecting agent of step (g) is piperidine.

8. The process of any one of the preceding clauses, wherein the polar aprotic solvent of step (a) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

9. The process of any one of the preceding clauses, wherein the polar aprotic solvent of step (d) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

10. The process of any one of the preceding clauses, wherein the polar aprotic solvent of step (e) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

11. The process of any one of the preceding clauses, wherein the polar aprotic solvent of step (f) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

12. The process of any one of the preceding clauses, wherein the polar aprotic solvent of step (g) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

13. The process of any one of the preceding clauses, wherein the polar aprotic solvent of step (h) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

14. The process of any one of the preceding clauses, wherein the polar aprotic solvent of step (a) is dimethylformamide (DMF).

15. The process of any one of the preceding clauses, wherein the polar aprotic solvent of step (d) is dimethylformamide (DMF).

16. The process of any one of the preceding clauses, wherein the polar aprotic solvent of step (e) is dimethylformamide (DMF).

17. The process of any one of the preceding clauses, wherein the polar aprotic solvent of step (f) is dimethylformamide (DMF).

18. The process of any one of the preceding clauses, wherein the polar aprotic solvent of step (g) is dimethylformamide (DMF).

19. The process of any one of the preceding clauses, wherein the polar aprotic solvent of step (h) is dimethylformamide (DMF).

20. The process of any one of the preceding clauses, wherein the base of step (b) is selected from the group consisting of N,N-Diisopropylethylamine (ⁱPr₂NEt), triethylamine (TEA), 4-methylmorpholine (NMM), imidazole, pyridine, and collidine.

21. The process of any one of the preceding clauses, wherein the base of step (d) is selected from the group consisting of N,N-Diisopropylethylamine (ⁱPr₂NEt), triethylamine (TEA), 4-methylmorpholine (NMM), imidazole, pyridine, and collidine.

22. The process of any one of the preceding clauses, wherein the base of step (f) is selected from the group consisting of N,N-Diisopropylethylamine (ⁱPr₂NEt), triethylamine (TEA), 4-methylmorpholine (NMM), imidazole, pyridine, and collidine.

23. The process of any one of the preceding clauses, wherein the base of step (h) is selected from the group consisting of N,N-Diisopropylethylamine (ⁱPr₂NEt), triethylamine (TEA), 4-methylmorpholine (NMM), imidazole, pyridine, and collidine.

24. The process of any one of the preceding clauses, wherein the base of step (b) is N,N-Diisopropylethylamine (ⁱPr₂NEt).

25. The process of any one of the preceding clauses, wherein the base of step (d) is N,N-Diisopropylethylamine (ⁱPr₂NEt).

26. The process of any one of the preceding clauses, wherein the base of step (f) is N,N-Diisopropylethylamine (ⁱPr₂NEt).

27. The process of any one of the preceding clauses, wherein the base of step (h) is N,N-Diisopropylethylamine (ⁱPr₂NEt).

28. The process of any one of the preceding clauses, wherein the second deprotecting agent of step (c) is selected from the group consisting of trifluoroacetic acid (TFA), acetic acid, trifluoroethanol, hexafluoroisopropanol, dichloromethane, and combinations thereof.

29. The process of any one of the preceding clauses, wherein the second deprotecting agent of step (c) is trifluoroacetic acid (TFA) or a mixture of acetic acid and trifluoroethanol.

30. The process of any one of the preceding clauses, wherein the coupling agent of step (d) is selected from the group consisting of benzotriazol-1-yl-oxytripyrrolidinophosphonium hexafluorophosphate (PyBOP), 1-[Bis(dimethylamino)methylene]-1H-1,2,3-triazolo[4,5-b]pyridinium 3-oxid hexafluorophosphate (HATU), 2-(1H-Benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-chloro-4,6-dimethoxy-1,3,5-triazine (CDMT), N-[(5-Chloro-3-oxido-1H-benzotriazol-1-yl)-4-morpholinylmethylene]-N-methylmethanaminium hexafluorophosphate (HDMC), 1-Cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium; tetrafluoroborate (TATU), N,N,N,N-tetramethyl-S-(1-oxido-2-pyridyl)thiouronium tetrafluoroborate (TOTT), N-Ethoxycarbonyl-2-ethoxy-1,2-dihydroquinoline (EEDQ), 1-Propanephosphonic anhydride (T3P), and 4-(4,6-dimethoxy-1,3,5-triazin-2-yl)-4-methyl-morpholinium chloride (DMTMM).

31. The process of any one of the preceding clauses, wherein the coupling agent of step (f) is selected from the group consisting of benzotriazol-1-yl-oxytripyrrolidinophosphonium hexafluorophosphate (PyBOP), 1-[Bis(dimethylamino)methylene]-1H-1,2,3-triazolo[4,5-b]pyridinium 3-oxid hexafluorophosphate (HATU), 2-(1H-Benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-chloro-4,6-dimethoxy-1,3,5-triazine (CDMT), N-[(5-Chloro-3-oxido-1H-benzotriazol-1-yl)-4-morpholinylmethylene]-N-methylmethanaminium hexafluorophosphate (HDMC), 1-Cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium; tetrafluoroborate (TATU), N,N,N′,N′-tetramethyl-S-(1-oxido-2-pyridyl)thiouronium tetrafluoroborate (TOTT), N-Ethoxycarbonyl-2-ethoxy-1,2-dihydroquinoline (EEDQ), 1-Propanephosphonic anhydride (T3P), and 4-(4,6-dimethoxy-1,3,5-triazin-2-yl)-4-methyl-morpholinium chloride (DMTMM).

32. The process of any one of the preceding clauses, wherein the coupling agent of step (h) is selected from the group consisting of benzotriazol-1-yl-oxytripyrrolidinophosphonium hexafluorophosphate (PyBOP), 1-[Bis(dimethylamino)methylene]-1H-1,2,3-triazolo[4,5-b]pyridinium 3-oxid hexafluorophosphate (HATU), 2-(1H-Benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-chloro-4,6-dimethoxy-1,3,5-triazine (CDMT), N-[(5-Chloro-3-oxido-1H-benzotriazol-1-yl)-4-morpholinylmethylene]-N-methylmethanaminium hexafluorophosphate (HDMC), 1-Cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium; tetrafluoroborate (TATU), N,N,N′,N′-tetramethyl-S-(1-oxido-2-pyridyl)thiouronium tetrafluoroborate (TOTT), N-Ethoxycarbonyl-2-ethoxy-1,2-dihydroquinoline (EEDQ), 1-Propanephosphonic anhydride (T3P), and 4-(4,6-dimethoxy-1,3,5-triazin-2-yl)-4-methyl-morpholinium chloride (DMTMM).

33. The process of any one of the preceding clauses, wherein the coupling agent of step (d) is benzotriazol-1-yl-oxytripyrrolidinophosphonium hexafluorophosphate (PyBOP).

34. The process of any one of the preceding clauses, wherein the coupling agent of step (f) is benzotriazol-1-yl-oxytripyrrolidinophosphonium hexafluorophosphate (PyBOP).

35. The process of any one of the preceding clauses, wherein the coupling agent of step (h) is benzotriazol-1-yl-oxytripyrrolidinophosphonium hexafluorophosphate (PyBOP).

36. The process of any one of the preceding clauses, wherein the third deprotecting agent of step (i) is selected from the group consisting of trifluoroacetic acid (TFA), hydrofluoric acid (HF), triflic acid (TfOH), a trifluoroacetic acid (TFA)/water/triisopropylsilane mixture, a trifluoroacetic acid (TFA)/dichloromethane (DCM) mixture, triethylsaline (TES), indole, a phenol/anisole mixture, and thioanisole.

37. The process of any one of the preceding clauses, wherein the third deprotecting agent of step (i) is a trifluoroacetic acid (TFA)/water/triisopropylsilane mixture.

38. The process of any one of the preceding clauses, wherein the cleavage reagent of step (i) is trifluoroacetic acid (TFA) or a trifluoroacetic acid (TFA)/water/triisopropylsilane mixture.

39. The process of any one of the preceding clauses, wherein the cleavage reagent of step (i) is a trifluoroacetic acid (TFA)/water/triisopropylsilane mixture.

40. The process of any one of the preceding clauses, where in the organic solvent of step (b) is selected from the group consisting of CH₂Cl₂, THF, diethyl ether, MeOH, EtOH, acetone, acetonitrile.

41. The process of any one of the preceding clauses, where in the organic solvent of step (c) is selected from the group consisting of CH₂Cl₂, THE, diethyl ether, MeOH, EtOH, acetone, acetonitrile.

42. The process of any one of the preceding clauses, where in the organic solvent of step (i) is selected from the group consisting of CH₂Cl₂, THF, diethyl ether, MeOH, EtOH, acetone, acetonitrile.

43. The process of any one of the preceding clauses, where in the organic solvent of step (b) is CH₂Cl₂.

44. The process of any one of the preceding clauses, where in the organic solvent of step (c) is CH₂Cl₂.

45. The process of any one of the preceding clauses, where in the organic solvent of step (i) is CH₂Cl₂.

46. The process of any one of the preceding clauses, wherein PG¹ is 9-Fluorenylmethyl-carbonyl (Fmoc) or t-butylcarbonyl (Boc).

47. The process of any one of the preceding clauses, wherein PG¹ is 9-Fluorenylmethyl-carbonyl (Fmoc).

48. The process of any one of the preceding clauses, wherein PG² is monomethoxytrityl (MMt) or 4-methyltrityl (Mtt).

49. The process of any one of the preceding clauses, wherein PG² is 4-methyltrityl (Mtt).

50. The process of any one of the preceding clauses, wherein LG is

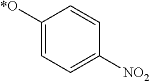

wherein, * represents a point of attachment to the rest of the compound.

51. The process of any one of the preceding clauses, wherein R¹ is t-butyl.

52. A process for preparing a compound of the formula 1

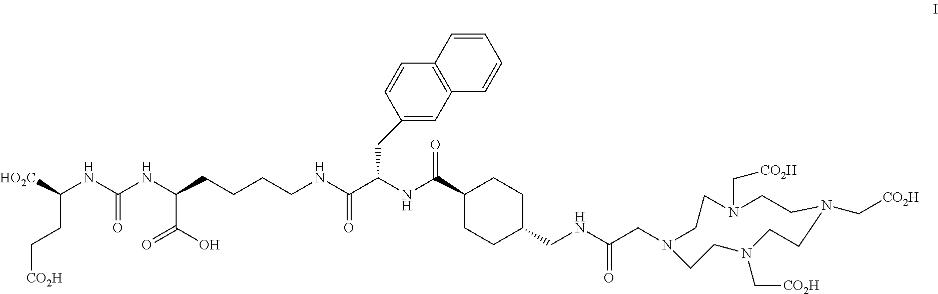

comprising one or more of

a. contacting a resin-based compound of the formula 1

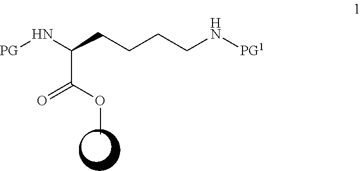

with a first deprotecting agent in the presence of a solvent to provide a resin-based compound of the formula 2

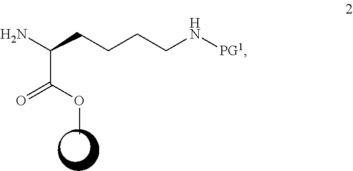

or

b. contacting the resin-based compound of the formula 2 with a compound of the formula 3

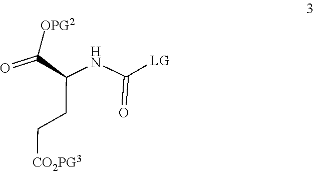

in the presence of an organic solvent and a base to provide a resin-based compound of the formula 4

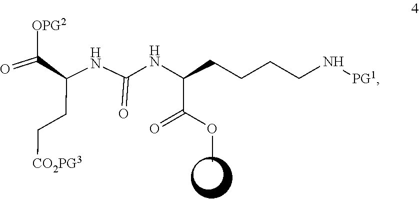

or

c. contacting the resin-based compound of the formula 4 with a second deprotecting agent in the presence of an organic solvent to provide a resin-based compound of the formula 5

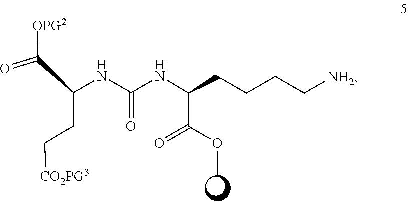

or

d. contacting the resin-based compound of the formula 5 with a compound of the formula 6

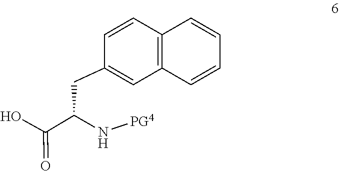

in the presence of a solvent, a coupling agent, and optionally a base and/or an additive to provide a resin-based compound of the formula 7

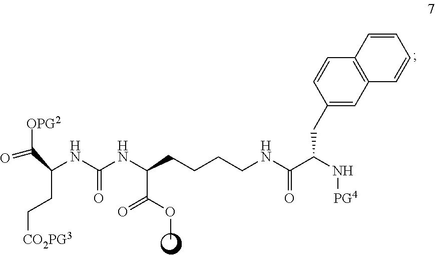

or

e. contacting the resin-based compound of the formula 7 with a third deprotecting agent in the presence of a solvent to provide a resin-based compound of the formula 8

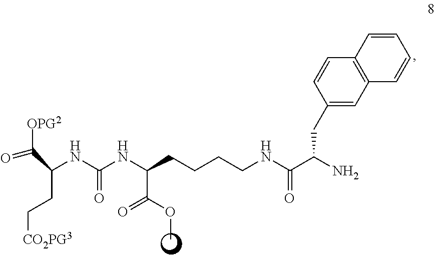

or

f. contacting the resin-based compound of the formula 8 with a compound of the formula 9

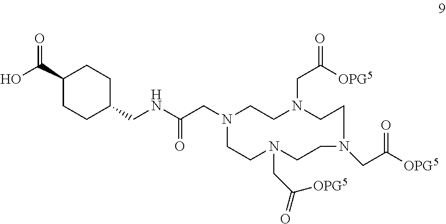

in the presence of a solvent, a coupling agent, and optionally a base and/or an additive to provide a resin-based compound of the formula 10

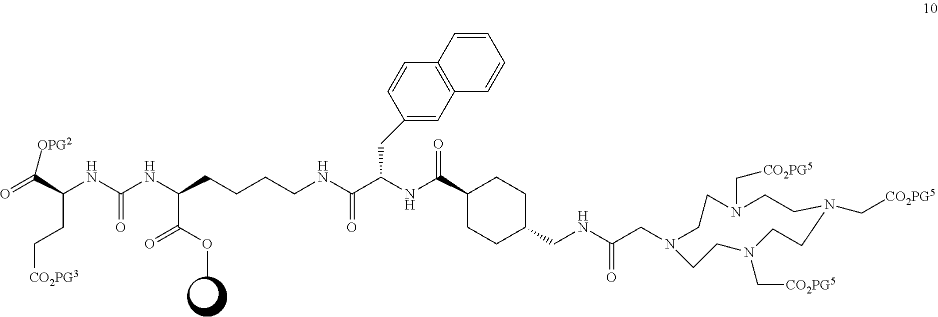

or

g. contacting the resin-based compound of the formula 10 with at least one third deprotecting agent and/or a cleavage reagent in the presence of an organic solvent to provide the compound of the formula I,

wherein PG, PG¹ and PG⁴ are amine protecting groups, PG², PG³, and PG⁵ are carboxyl protecting groups, and LG in compound C is a leaving group.

53. The process of clause 52, wherein PG, PG¹ and PG⁴ are selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt).

54. The process of clause 52 or 53, wherein PG is 9-fluorenylmethyl-carbonyl.

55. The process of any one of clauses 52 to 54, wherein PG¹ is monomethoxytrityl (MMt) or 4-methyltrityl (Mtt).

56. The process of any one of clauses 52 to 55, wherein PG¹ is 4-methyltrityl (Mtt).

57. The process of any one of clauses 52 to 56, wherein PG⁴ is 9-fluorenylmethyl-carbonyl.

58. The process of any one of clauses 52 to 57, wherein PG², PG³, and PG⁵ are each t-butyl.

59. The process of any one of clauses 52 to 58, wherein LG in compound C is s selected from imidazolyl, 4-nitrophenoxy, and phenoxy.

60. The process of any one of clauses 52 to 59, wherein the first deprotecting agent of step (a) comprises a reagent or mixture of reagents capable of removing a protecting group selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt).

61. The process of any one of clauses 52 to 60, wherein the first deprotecting agent of step (a) comprises a reagent or mixture of reagents capable of removing a 9-fluorenylmethyl-carbonyl.

62. The process of any one of clauses 52 to 61, wherein the first deprotecting agent of step (a) is selected from the group consisting of piperidine, morpholine, 1,8-diazabicyclo[5.4.0]undec-7-ene (DBU), diethylamine (DEA), dicyclohexamine, 4-methylpiperidine (4MP), tris (2-aminoethyl)amine, pyridine, and collidine.

63. The process of any one of clauses 52 to 62, wherein the first deprotecting agent of step (a) is piperidine.

64. The process of any one of clauses 52 to 63, wherein the solvent of step (a) is a polar aprotic solvent.

65. The process of any one of clauses 52 to 64, wherein the solvent of step (a) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

66. The process of any one of clauses 52 to 65, wherein the base of step (b) is selected from the group consisting of N,N-diisopropylethylamine (ⁱPr₂NEt), triethylamine (TEA), 4-methylmorpholine (NMM), imidazole, pyridine, and collidine.

67. The process of any one of clauses 52 to 66, wherein the organic solvent of step (b) is selected from the group consisting of CH₂Cl₂, THF, diethyl ether, MeOH, EtOH, acetone, and acetonitrile.

68. The process of any one of clauses 52 to 67, wherein the second deprotecting agent of step (c) comprises a reagent or mixture of reagents capable of removing a protecting group selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt).

69. The process of any one of clauses 52 to 68, wherein the second deprotecting agent of step (c) comprises a reagent or mixture of reagents capable of removing a 4-methyltrityl (Mtt).

70. The process of any one of clauses 52 to 69, wherein the second deprotecting agent of step (c) is selected from the group consisting of trifluoroacetic acid (TFA), acetic acid, 2,2,2-trifluoroethanol, hexafluoroisopropanol, and combinations thereof.

71. The process of any one of clauses 52 to 70, wherein the second deprotecting agent of step (c) is a mixture of acetic acid and 2,2,2-trifluoroethanol.

72. The process of any one of clauses 52 to 71, wherein the organic solvent of step (c) is selected from the group consisting of CH₂Cl₂, THF, diethyl ether, MeOH, EtOH, acetone, and acetonitrile.

73. The process of any one of clauses 52 to 72, wherein the coupling agent of step (d) is selected from the group consisting of benzotriazol-1-yloxy-tris (dimethylamino)-phosphonium hexafluorophosphate (BOP), benzotriazol-1-yl-oxytripyrrolidinophosphonium hexafluorophosphate (PyBOP), 7-aza-benzotriazol-1-yloxy-tripyrrolidino-phosphonium hexafluorophosphate (PyAOP), ethyl cyano (hydroxyimino) acetato-O₂)-tri-(1-pyrrolidinyl)-phosphonium hexafluorophosphate (PyOxim), bromo-tripyrrolidino-phosphonium hexa-fluorophosphate (PyBrOP), 3-(diethoxy-phosphoryloxy)-1,2,3-benzo[d]triazin-4 (3H)-one (DEPBT), 1-[bis(dimethylamino)methylene]-1H-1,2,3-triazolo[4,5-b]pyridinium 3-oxid hexafluorophosphate (HATU), 2-(1H-benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-(6-chloro-1H-benzotriazol-1-yl)-N,N,N′,N′-tetramethylaminium hexafluorophosphate (HCTU), 2-chloro-4,6-dimethoxy-1,3,5-triazine (CDMT), N-[(5-chloro-3-oxido-1H-benzotriazol-1-yl)-4-morpholinylmethylene]-N-methylmethanaminium hexafluorophosphate (HDMC), 1-cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium tetrafluoroborate (TATU), N,N,N′,N-tetramethyl-S-(1-oxido-2-pyridyl)thiouronium tetrafluoroborate (TOTT), tetramethylfluoroformamidinium hexafluorophosphate (TFFH), N-ethoxycarbonyl-2-ethoxy-1,2-dihydroquinoline (EEDQ), 1-propanephosphonic anhydride (T3P), 4-(4,6-dimethoxy-1,3,5-triazin-2-yl)-4-methyl-morpholinium chloride (DMTMM), dicyclohexylcarbodiimide (DCC), diisopropylcarbodiimide (DIC), (N-(3-dimethylaminopropyl)-N′-ethylcarbodiimide (EDAC), and 1,1′-carbonyldimidizole (CDI).

74. The process of any one of clauses 52 to 73, wherein the base of step (d) is selected from the group consisting of N,N-diisopropylethylamine (ⁱPr₂NEt), triethylamine (TEA), 4-methylmorpholine (NMM), imidazole, pyridine, and collidine.

75. The process of any one of clauses 52 to 74, wherein the additive in step (d), when present, is selected from the group consisting of 1-hydroxybenzotriazole (HOBt), 1-hydroxybenzotriazole-6-sulfonamidomethyl resin HCl (HOBt-6-sulfonamidomethyl resin HCl), hydroxy-3,4-dihydro-4-oxo-1,2,3-benzotriazine (HOOBt), N-hydroxysuccinimide (NHS), 1-hydroxy-7-aza-1H-benzotriazole (HOAt), ethyl 2-cyano-2-(hydroximino)acetate, and 4-(N,N-dimethylamino)pyridine (DMAP).

76. The process of any one of clauses 52 to 75, wherein the solvent of step (d) is a polar aprotic solvent.

77. The process of any one of clauses 52 to 76, wherein the solvent of step (d) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

78. The process of any one of clauses 52 to 77, wherein the third deprotecting agent of step (e) comprises a reagent or mixture of reagents capable of removing a protecting group selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt).

79. The process of any one of clauses 52 to 78, wherein the third deprotecting agent of step (e) comprises a reagent or mixture of reagents capable of removing a 9-fluorenylmethyl-carbonyl.

80. The process of any one of clauses 52 to 79, wherein the first deprotecting agent of step (e) is selected from the group consisting of piperidine, morpholine, 1,8-diazabicyclo[5.4.0]undec-7-ene (DBU), diethylamine (DEA), dicyclohexamine, 4-methylpiperidine (4MP), tris (2-aminoethyl)amine, pyridine, and collidine.

81. The process of any one of clauses 52 to 80, wherein the first deprotecting agent of step (e) is piperidine.

82. The process of any one of clauses 52 to 81, wherein the solvent of step (e) is a polar aprotic solvent.

83. The process of any one of clauses 52 to 82, wherein the solvent of step (e) is selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

84. The process of any one of clauses 52 to 83, wherein the coupling agent of step (f) is selected from the group consisting of benzotriazol-1-yloxy-tris (dimethylamino)-phosphonium hexafluorophosphate (BOP), benzotriazol-1-yl-oxytripyrrolidinophosphonium hexafluorophosphate (PyBOP), 7-aza-benzotriazol-1-yloxy-tripyrrolidino-phosphonium hexafluorophosphate (PyAOP), ethyl cyano (hydroxyimino) acetato-O₂)-tri-(1-pyrrolidinyl)-phosphonium hexafluorophosphate (PyOxim), bromo-tripyrrolidino-phosphonium hexa-fluorophosphate (PyBrOP), 3-(diethoxy-phosphoryloxy)-1,2,3-benzo[d]triazin-4 (3H)-one (DEPBT), 1-[bis(dimethylamino)methylene]-1H-1,2,3-triazolo[4,5-b]pyridinium 3-oxid hexafluorophosphate (HATU), 2-(1H-benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-(6-chloro-1H-benzotriazol-1-yl)-N,N,N′,N′-tetramethylaminium hexafluorophosphate (HCTU), 2-chloro-4,6-dimethoxy-1,3,5-triazine (CDMT), N-[(5-chloro-3-oxido-1H-benzotriazol-1-yl)-4-morpholinylmethylene]-N-methylmethanaminium hexafluorophosphate (HDMC), 1-cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium tetrafluoroborate (TATU), N,N,N,N-tetramethyl-S-(1-oxido-2-pyridyl)thiouronium tetrafluoroborate (TOTT), tetramethylfluoroformamidinium hexafluorophosphate (TFFH), N-ethoxycarbonyl-2-ethoxy-1,2-dihydroquinoline (EEDQ), 1-propanephosphonic anhydride (T3P), 4-(4,6-dimethoxy-1,3,5-triazin-2-yl)-4-methyl-morpholinium chloride (DMTMM), dicyclohexylcarbodiimide (DCC), diisopropylcarbodiimide (DIC), (N-(3-dimethylaminopropyl)-N′-ethylcarbodiimide (EDAC), and 1,1′-carbonyldimidizole (CDI).

85. The process of any one of clauses 52 to 84, wherein the base in step (f), when present, is selected from the group consisting of N,N-diisopropylethylamine (ⁱPr₂NEt), triethylamine (TEA), 4-methylmorpholine (NMM), imidazole, pyridine, and collidine.

86. The process of any one of clauses 52 to 85, wherein the additive in step (f), when present, is selected from the group consisting of 1-hydroxybenzotriazole (HOBt), 1-hydroxybenzotriazole-6-sulfonamidomethyl resin HCl (HOBt-6-sulfonamidomethyl resin HCl), hydroxy-3,4-dihydro-4-oxo-1,2,3-benzotriazine (HOOBt), N-hydroxysuccinimide (NHS), 1-hydroxy-7-aza-1H-benzotriazole (HOAt), ethyl 2-cyano-2-(hydroximino)acetate, and 4-(N,N-dimethylamino)pyridine (DMAP).

87. The process of any one of clauses 52 to 86, wherein the solvent in step (f) is a polar aprotic solvent.

88. The process of any one of clauses 52 to 87, wherein the solvent in step (f) is a polar aprotic solvent selected from the group consisting of dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO).

89. The process of any one of clauses 52 to 88, wherein the third deprotecting agent of step (g) is a reagent or mixture of reagents capable of removing a protecting group selected from the group consisting of t-butyl, benzyl, 2-chlorotrityl (2-Cl-Trt), 2,4-dimethoxybenzyl (Dmb), allyl, 1,1-dimethylallyl (Dma), and p-nitrobenzyl (pNB).

90. The process of any one of clauses 52 to 89, wherein the third deprotecting agent of step (g) is selected from the group consisting of trifluoroacetic acid (TFA), hydrofluoric acid (HF), triflic acid (TfOH), a trifluoroacetic acid (TFA)/water/triisopropylsilane mixture, a trifluoroacetic acid (TFA)/dichloromethane (DCM) mixture, triethylsaline (TES), indole, a phenol/anisole mixture, and thioanisole.

91. The process of any one of clauses 52 to 90, wherein the third deprotecting agent of step (g) is a trifluoroacetic acid (TFA)/water/triisopropylsilane mixture.

92. The process of any one of clauses 52 to 91, wherein the cleavage reagent of step (g) is trifluoroacetic acid (TFA) or a trifluoroacetic acid (TFA)/water/triisopropylsilane mixture.

93. The process of any one of clauses 52 to 92, wherein the cleavage reagent of step (g) is a trifluoroacetic acid (TFA)/water/triisopropylsilane mixture.

94. The process of any one of clauses 52 to 93, wherein the organic solvent of step (g) is selected from the group consisting of CH₂Cl₂, THE, diethyl ether, MeOH, EtOH, acetone, and acetonitrile.

95. The process of any one of clauses 52 to 94, wherein the compound of the formula 6, a solvent, a coupling agent, and optionally a base and/or an additive are combined to form a mixture comprising an activated compound of the formula 6′

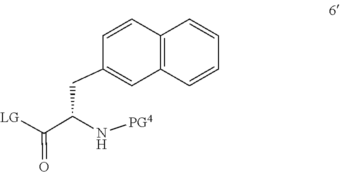

wherein LG in compound 6′ is a leaving group, prior to contacting with the resin-based compound of the formula 5.

96. The process of any one of clauses 52 to 94, wherein the compound of the resin-based compound of the formula 5, the compound of the formula 6, a solvent, a coupling agent, and optionally a base and/or an additive are contacted together prior to or simultaneous with the in situ formation of an activated compound of the formula 6′

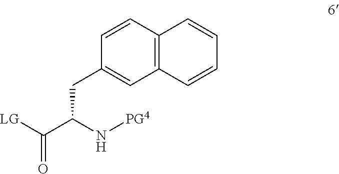

wherein LG in compound 6′ is a leaving group.

97. The process of clause 95 or 96, wherein LG in compound 6′ is selected from the group consisting of 1-(λ¹-oxidaneyl)-1H-benzo[d][1,2,3]triazole, 3-(λ¹-oxidaneyl)-3H-[1,2,3]triazolo[4,5-b]pyridine, ethyl cyano (hydroxyimino)acetate (Oxyma), bromide, 3-(λ¹-oxidaneyl)benzo[d][1,2,3]triazin-4 (3H)-one, 6-chloro-1-(λ¹-oxidaneyl)-1H-benzo[d][1,2,3]triazole, 2,4-dimethoxy-6-(λ¹-oxidaneyl)-1,3,5-triazine, etabonic acid, propylphosphonic acid, dicyclohexylurea, 1-(3-(dimethyl-λ⁴-azaneyl) propyl)-3-ethylurea, and imidazolyl.

98. The process of any one of the clauses 95 to 97, wherein LG in compound 6′ is imidazolyl.

99. The process of any one of the preceding clauses, wherein the compound of the formula 9, a solvent, a coupling agent, and optionally a base and/or an additive are combined to form a mixture comprising an activated compound of the formula 9′

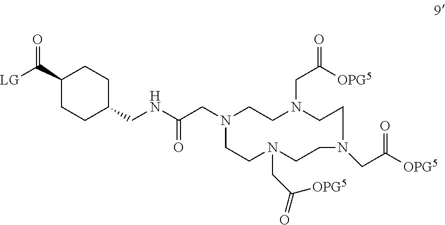

wherein LG in compound 9′ is a leaving group, prior to contacting with the resin-based compound of the formula H.

100. The process of any one of clauses 52 to 98, wherein the resin-based compound of the formula 8, the compound of the formula 9, a solvent, a coupling agent, and optionally a base and/or an additive are contacted together prior to or simultaneous with the in situ formation of an activated compound of the formula 9′

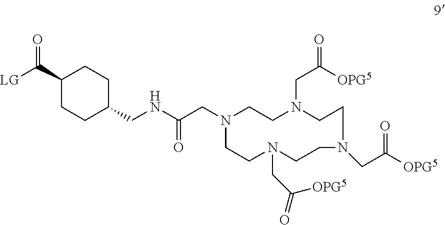

wherein LG in compound 9′ is a leaving group.

101. The process of clause 99 or 100, wherein LG in compound 9′ is selected from the group consisting of 1-(λ¹-oxidaneyl)-1H-benzo[d][1,2,3]triazole, 3-(λ¹-oxidaneyl)-3H-[1,2,3]triazolo[4,5-b]pyridine, ethyl cyano (hydroxyimino)acetate (Oxyma), bromide, 3-(λ¹-oxidaneyl)benzo[d][1,2,3]triazin-4 (3H)-one, 6-chloro-1-(λ¹-oxidaneyl)-1H-benzo[d][1,2,3]triazole, 2,4-dimethoxy-6-(λ¹-oxidaneyl)-1,3,5-triazine, etabonic acid, propylphosphonic acid, dicyclohexylurea, 1-(3-(dimethyl-λ⁴-azaneyl) propyl)-3-ethylurea, and imidazolyl.

102. The process of any one of the clauses 99 to 101, wherein LG in compound 9′ is imidazolyl.

103. A resin-based compound of the formula 4

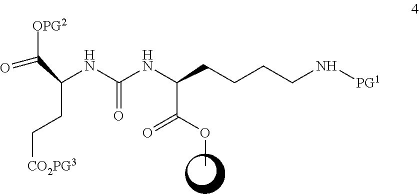

wherein PG¹ is an amine protecting group, and PG² and PG³ are each independently a carboxyl protecting group.

104. A resin-based compound of the formula 5

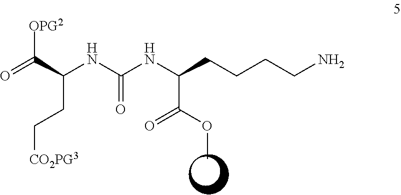

wherein PG² and PG³ are each independently a carboxyl protecting group.

105. A resin-based compound of the formula 7

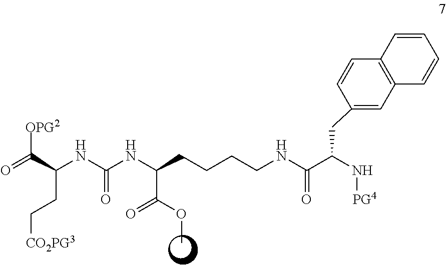

wherein PG² and PG³ are each independently a carboxyl protecting group, and PG⁴ is an amine protecting group.

106. A resin-based compound of the formula 8

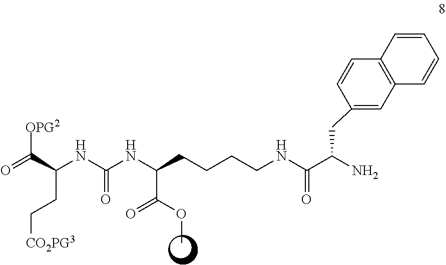

wherein PG² and PG³ are each independently a carboxyl protecting group.

107. A resin-based compound of the formula 10

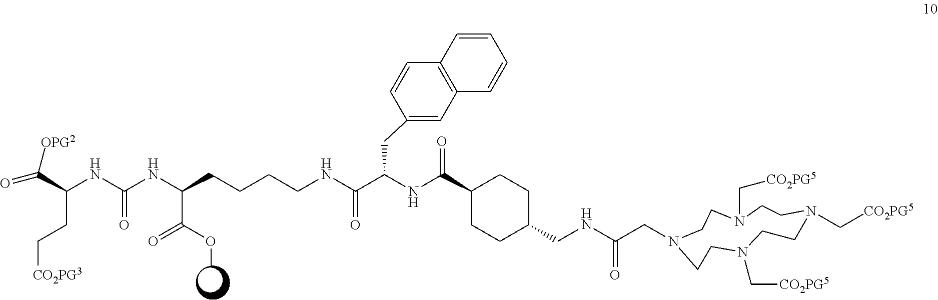

wherein each PG², PG³, and PG⁵ is independently a carboxyl protecting group.

108. The resin-based compound of any one of clauses 103 to 107, wherein PG¹ and PG⁴, when present, are independently selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt).

109. The resin-based compound of any one of clauses 103 to 108, wherein PG¹, when present, is monomethoxytrityl (MMt) or 4-methyltrityl (Mtt).

110. The resin-based compound of any one of clauses 103 to 109, wherein PG¹, when present, is 4-methyltrityl (Mtt).

111. The resin-based compound of any one of clauses 103 to 110, wherein PG⁴, when present, is 9-fluorenylmethyl-carbonyl.

112. The resin-based compound of any one of clauses 103 to 111, wherein PG², PG³, and PG⁵, when present, are each t-butyl.

113. A resin-based compound of the formula C

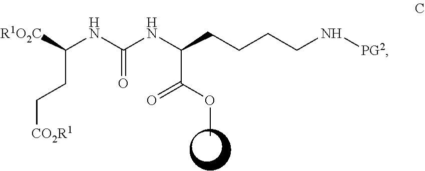

wherein PG² is an amine protecting group, and each R¹ is a C₁-C₄ alkyl or a cyclohexyl. 114. A resin-based compound of the formula C′

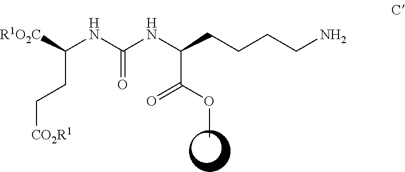

wherein each R¹ is a C₁-C₄ alkyl or a cyclohexyl.

115. A resin-based compound of the formula D

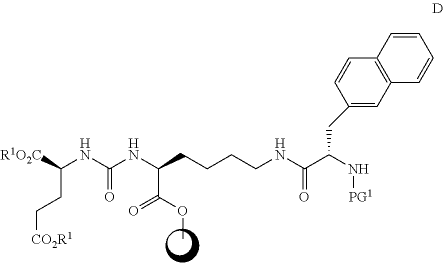

wherein PG¹ is an amine protecting groups, and each R¹ is a C₁-C₄ alkyl or a cyclohexyl.

116. A resin-based compound of the formula D′

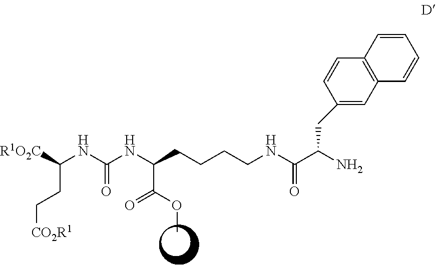

wherein each R¹ is a C₁-C₄ alkyl or a cyclohexyl.

117. A resin-based compound of the formula E

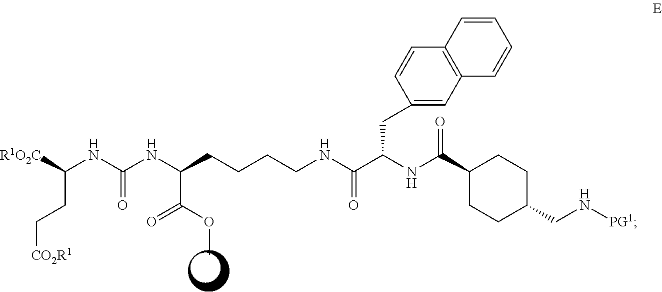

wherein PG¹ is an amine protecting groups, and each R¹ is a C₁-C₄ alkyl or a cyclohexyl.

118. A resin-based compound of the formula E′

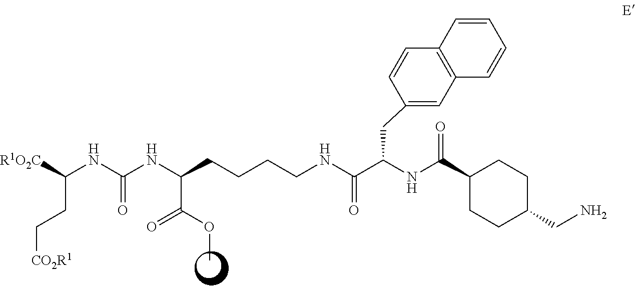

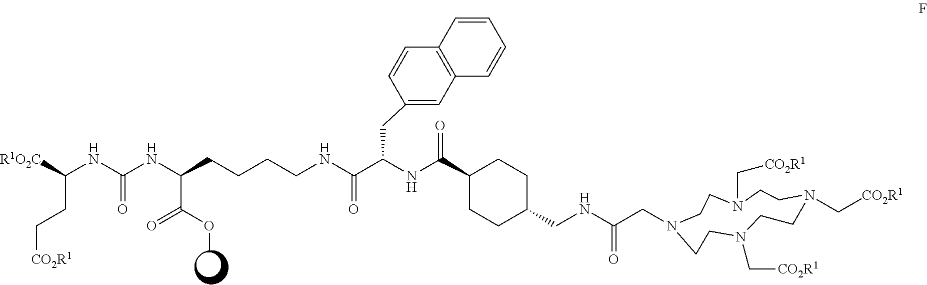

wherein each R¹ is a C₁-C₄ alkyl or a cyclohexyl.

119. A resin-based compound of the formula F

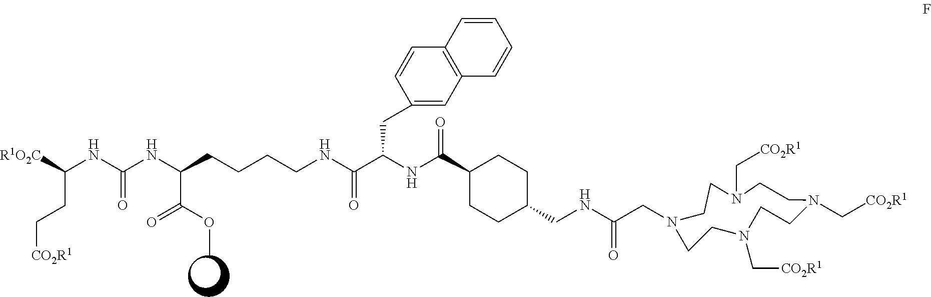

each R¹ is a C₁-C₄ alkyl or a cyclohexyl.

120. The resin-based compound of clause 115 or 117, wherein PG¹ is 9-Fluorenylmethyl-carbonyl (Fmoc) or 1-butylcarbonyl (Boc).

121. The resin-based compound of any one of clauses 115, 117, or 120, wherein PG¹ is 9-Fluorenylmethyl-carbonyl (Fmoc).

122. The resin-based compound of clause 113, wherein PG² is monomethoxytrityl (MMt) or 4-methyltrityl (Mtt).

123. The resin-based compound of clause 113 or 122, wherein PG² is 4-methyltrityl (Mtt).

124. The resin-based compound of any one of clauses 113 to 119, wherein R¹ is t-butyl.

### Definitions

As used herein, the term “alkyl” includes a chain of carbon atoms, which is optionally branched and contains from 1 to 4 carbon atoms, and the like may be referred to as “lower alkyl.” Illustrative alkyl groups include, but are not limited to, methyl, ethyl, n-propyl, isopropyl, n-butyl, isobutyl, sec-butyl, and tert-butyl.

As used herein, unless otherwise indicated, the term “abnormal cell growth” refers to cell growth that is independent of normal regulatory mechanisms (e.g., loss of contact inhibition).

The term “subject” refers to a mammalian patient in need of such treatment, such as a human.

As used herein, the term “protecting group” or “PG” refers to any group as commonly known to one of ordinary skill in the art that can be introduced into a molecule by chemical modification of a functional group, such as an amine or hydroxyl, to obtain chemoselectivity in a subsequent chemical reaction. It will be appreciated that such protecting groups can be subsequently removed from the functional group at a later point in a synthesis to provide further opportunity for reaction at such functional groups or, in the case of a final product, to unmask such functional group. Protecting groups have been described in, for example, Wuts, P. G. M., Greene, T. W., Greene, T. W., & John Wiley & Sons. (2006). Greene's protective groups in organic synthesis. Hoboken, N.J: Wiley-Interscience. One of skill in the art will readily appreciate the chemical process conditions under which such protecting groups can be installed on a functional group. Suitable amine protecting groups useful in connection with the present disclosure include, but are not limited to, 9-Fluorenylmethyl-carbonyl (Fmoc) and 1-butylcarbonyl (Boc).

As used herein, the term “leaving group” of “LG” refers to any group commonly known to one of ordinary skill in the art that departs with a pair of electrons in heterolytic bond cleavage. Leaving groups can be anions or neutral molecules, but in either case it is crucial that the leaving group be able to stabilize the additional electron density that results from bond heterolysis.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

### Synthesis Methods

### Method 1:

In some embodiments, the disclosure provides a process for preparing a compound of the formula I

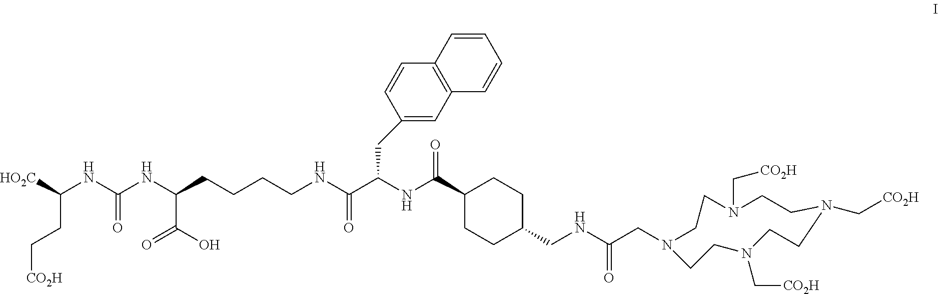

comprising

a. contacting a resin-based compound of the formula A

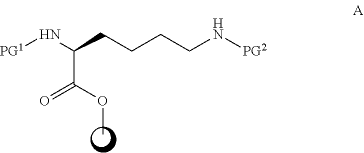

with a first deprotecting agent in a polar aprotic solvent to provide a resin-based compound of the formula A′

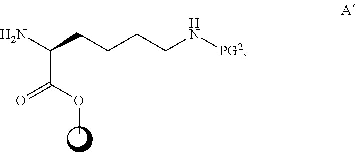

or

b. contacting the resin-based compound of the formula A′ with a compound of the formula B

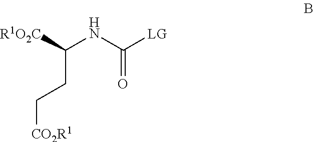

in the presence of an organic solvent and a base to provide a resin-based compound of the formula C

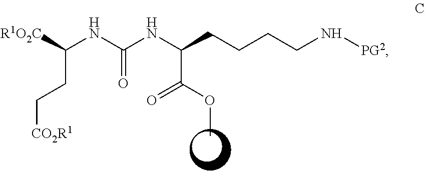

or

c. contacting the resin-based compound of the formula C with a second deprotecting agent in the presence of an organic solvent to provide a resin-based compound of the formula C′

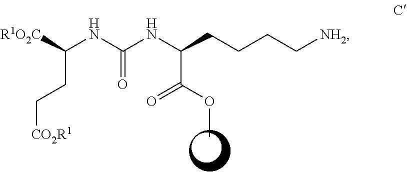

or

d. contacting the resin-based compound of the formula C′ with PG¹-3-(2-napthyl)-L-alanine in the presence of a polar aprotic solvent, a coupling agent, and a base to provide a resin-based compound of the formula D

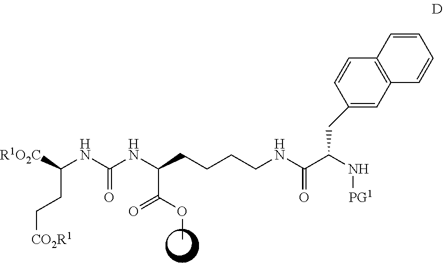

or

e. contacting the resin-based compound of the formula D with the first deprotecting agent in a polar aprotic solvent to provide a resin-based compound of the formula D′

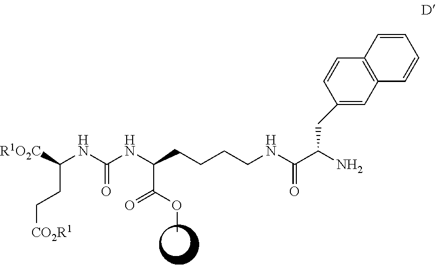

or

f. contacting the resin-based compound of the formula D′ with PG¹-tranexamic acid in the presence of a polar aprotic solvent, a coupling agent, and a base to provide a resin-based compound of the formula E

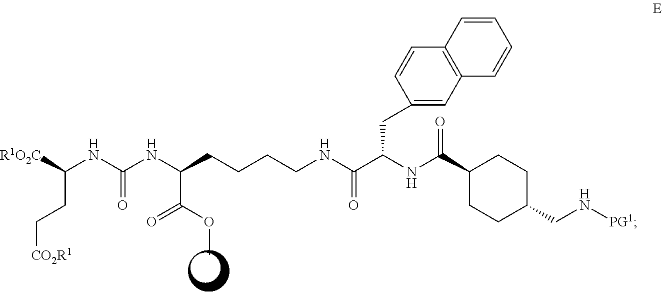

or

g. contacting the resin-based compound of the formula E with the first deprotecting agent in a polar aprotic solvent to provide a resin-based compound of the formula E′

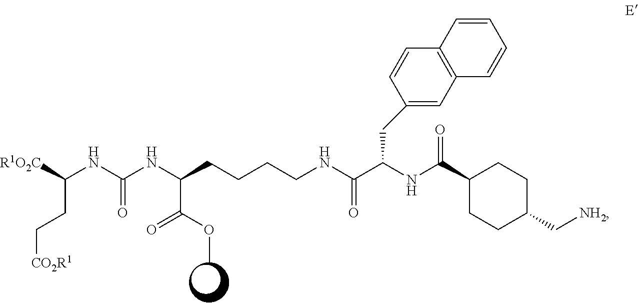

or

h. contacting the resin-based compound of the formula E′ with a compound of the formula E^{2′}

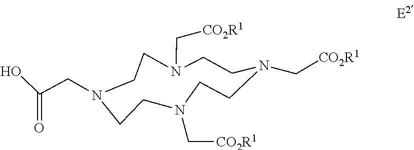

in the presence of a polar aprotic solvent, a coupling agent, and a base to provide a resin-based compound of the formula F

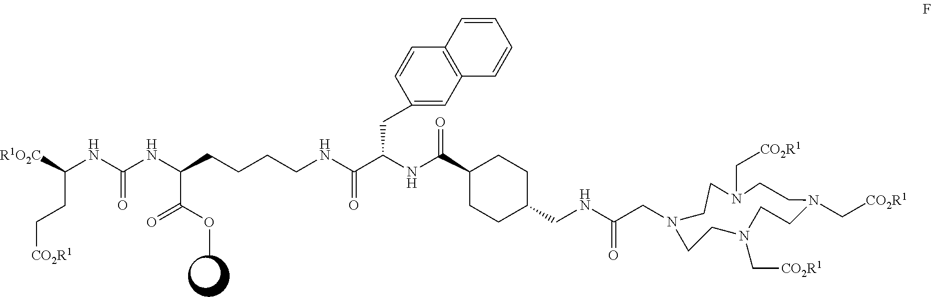

or

i. contacting the resin-based compound of the formula F with a third deprotecting agent and/or a cleavage reagent in an organic solvent,

wherein PG¹ and PG² are amine protecting groups, LG is a leaving group, and R¹ is a C₁-C₄ alkyl or a cyclohexyl.

### Method 2:

Alternatively, in some embodiments, the disclosure provides a process for preparing a compound of the formula 1

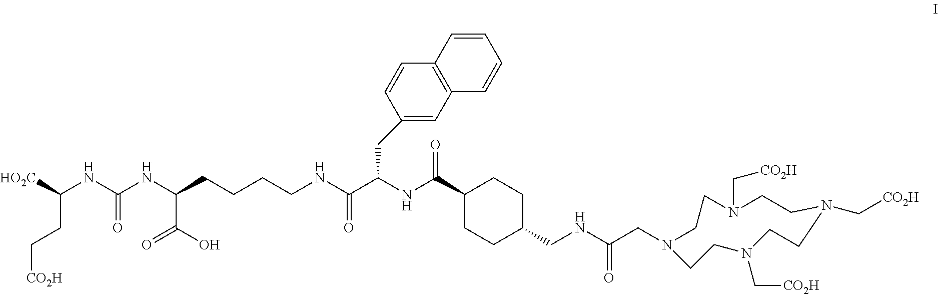

comprising one or more of

a. contacting a resin-based compound of the formula 1

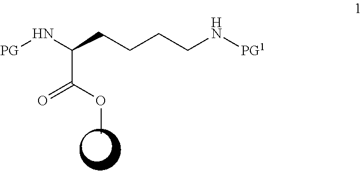

with a first deprotecting agent in the presence of a solvent to provide a resin-based compound of the formula 2

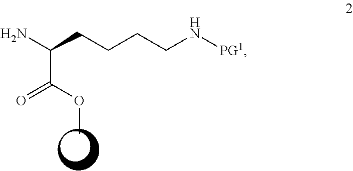

or

b. contacting the resin-based compound of the formula 2 with a compound of the formula 3

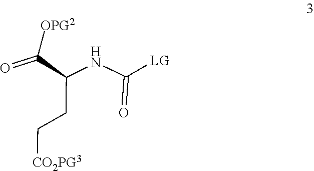

in the presence of an organic solvent and a base to provide a resin-based compound of the formula 4

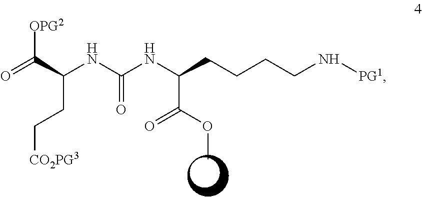

or

c. contacting the resin-based compound of the formula 4 with a second deprotecting agent in the presence of an organic solvent to provide a resin-based compound of the formula 5

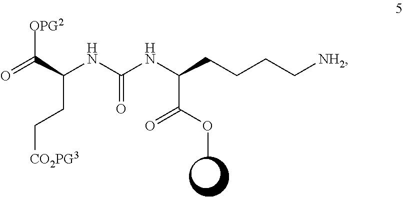

or

d. contacting the resin-based compound of the formula 5 with a compound of the formula 6

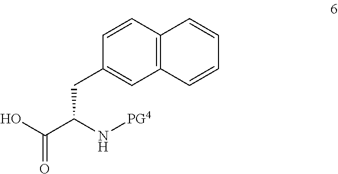

in the presence of a solvent, a coupling agent, and optionally a base and/or an additive to provide a resin-based compound of the formula 7

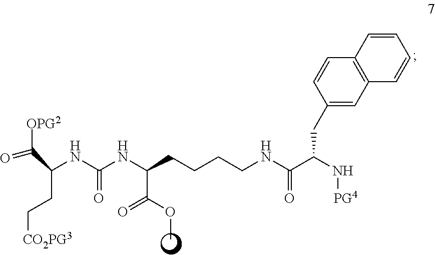

or

e. contacting the resin-based compound of the formula 7 with a third deprotecting agent in the presence of a solvent to provide a resin-based compound of the formula 8

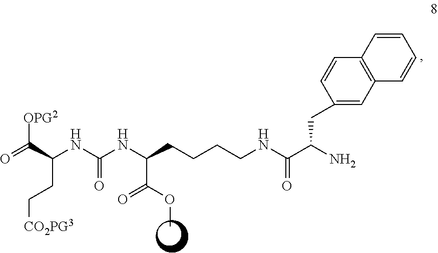

or

f. contacting the resin-based compound of the formula 8 with a compound of the formula 9

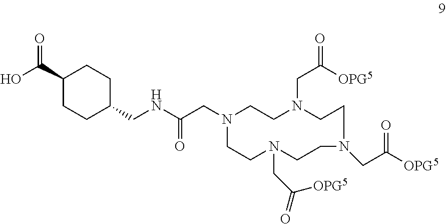

in the presence of a solvent, a coupling agent, and optionally a base and/or an additive to provide a resin-based compound of the formula 10

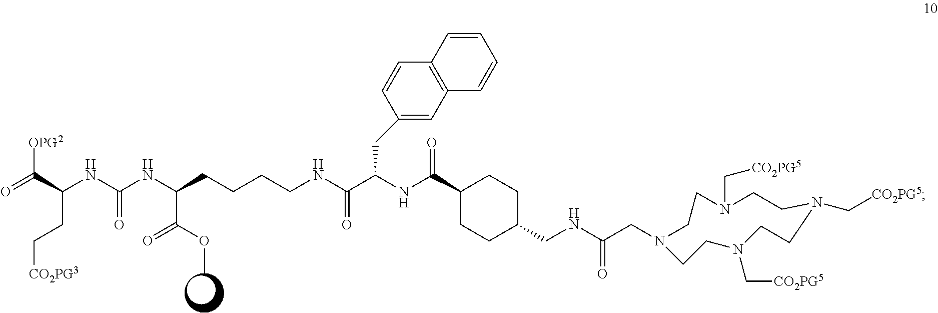

or

g. contacting the resin-based compound of the formula 10 with at least one third deprotecting agent and/or a cleavage reagent in the presence of an organic solvent to provide the compound of the formula I,

wherein PG, PG¹ and PG⁴ are amine protecting groups, PG², PG³, and PG⁵ are carboxyl protecting groups, and LG in compound C is a leaving group.

It will be appreciated that the present disclosure provides processes for preparing a compound of the formula I described in the paragraphs above (by Method 1 or Method 2), comprising more than one of the step listed in the alternative. Accordingly, the present disclosure provides a process for preparing a compound of the formula I, comprising steps (a) and (b). Alternatively, the present disclosure provides a process for preparing a compound of the formula I, comprising steps (b) and (c). Alternatively, the present disclosure provides a process for preparing a compound of the formula I, comprising steps (c) and (d). Alternatively, the present disclosure provides a process for preparing a compound of the formula I, comprising steps (d) and (e).

Alternatively, the present disclosure provides a process for preparing a compound of the formula I, comprising steps (e) and (f). Alternatively, the present disclosure provides a process for preparing a compound of the formula I, comprising steps (f) and (g). Alternatively, the present disclosure provides a process for preparing a compound of the formula I, comprising steps (g) and (h). Alternatively, the present disclosure provides a process for preparing a compound of the formula I, comprising steps (h) and (i). Alternatively, the present disclosure provides a process for preparing a compound of the formula I, comprising steps (a), (b) and (c). Alternatively, the present disclosure provides a process for preparing a compound of the formula I, comprising steps (b), (c) and (d). Alternatively, the present disclosure provides a process for preparing a compound of the formula I, comprising steps (c), (d) and (e). Alternatively, the present disclosure provides a process for preparing a compound of the formula I, comprising steps (d), (e) and (f). Alternatively, the present disclosure provides a process for preparing a compound of the formula I, comprising steps (e), (f) and (g). Alternatively, the present disclosure provides a process for preparing a compound of the formula I, comprising steps (d), (e) and (f). Alternatively, the present disclosure provides a process for preparing a compound of the formula I, comprising steps (f), (g) and (h). Alternatively, the present disclosure provides a process for preparing a compound of the formula I, comprising steps (d), (e) and (f). Alternatively, the present disclosure provides a process for preparing a compound of the formula I, comprising steps (g), (h) and (i). Alternatively, the present disclosure provides a process for preparing a compound of the formula I, comprising steps (a), (b), (c), (d), (e), (f), (g), and (h).

In the various embodiments described herein, it will be appreciated by a person having ordinary skill in the art that the choice of protecting groups used in the preparation of compound (I) can be chosen from various alternatives known in the art. It will further be appreciated that any protecting group scheme will be chosen such that the protecting groups used are an orthogonal protection strategy. As used herein, “orthogonal protection” is a strategy allowing for the deprotection of one or more protective groups among multiple protecting groups, where the protecting groups are removed one at a time each using a dedicated set of reaction conditions without affecting the others, or together as a subset of all protecting groups used in the synthesis using a dedicated set of reaction conditions without affecting the others.

In step (a) of Method 1, PG¹ on the resin-based compound of the formula A can be a protecting group, such as 9-Fluorenylmethyl-carbonyl (Fmoc) or 1-butylcarbonyl (Boc), and PG² can be an orthogonal protecting group, such as 4-methyltrityl (Mtt) or monomethoxytrityl (MMt). The first deprotecting agent in step (a) of Method 1 can be an acidic or basic deprotecting agent, depending on the identity of PG¹ and/or PG². In particular, the first deprotecting agent can be a basic deprotecting agent, such as piperidine. morpholine, 1,8-diazabicyclo[5.4.0]undec-7-ene (DBU), diethylamine (DEA), dicyclohexamine, 4-methylpiperidine (4MP), Tris(2-aminoethyl)amine, pyridine, or a collidine. In particular, the first deprotecting agent can be an acidic deprotecting agent, such as TFA, HCl, or H₃PO₄. Suitable deprotecting agents and conditions for step (a) of Method 1 can be found in for example, Wuts, P. G. M., Greene, T. W., Greene, T. W., & John Wiley & Sons. (2006). Greene's protective groups in organic synthesis. Hoboken, N.J: Wiley-Interscience. In step (a) of Method 1, the base used to remove the Fmoc group is piperidine. The polar-aprotic solvent of step (a) of Method 1 can be any polar-aprotic solvent known in the art. Suitable polar-aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO), and the like. It will be appreciated that step (a) of Method 1 can be conducted at any temperature commonly used in solid-phase synthesis, such as room temperature, under cooling, or under warming conditions. The deprotection step (a) of Method 1 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the deprotection step (a) of Method 1, the resin-based compound of the formula A′ produced in step (a) can be washed with reagents commonly known in the art, such as DMF, IPA, CH₂Cl₂, and the like.

In step (b) of Method 1, R¹ on the compound of the formula B can C₁-C₄ alkyl, preferably tert-butyl, or a cyclohexyl, and LG is a leaving group, such as an active ester. Suitable active esters known in the art include, but are not limited to 4-nitrophenyl, 2,4,5-trichlorophenyl, NHS ester, benzotriazyl, and the like. In some embodiments, the LG is a 4-nitrophenyl ester. It will be appreciated that the choice of R¹ will be orthogonal to the choice for PG¹, PG², and/or the resin bead connection. In step (b) of Method 1, the base can be any base commonly known in the art for urea formation reactions. Suitable bases include, but are not limited to, N,N-Diisopropylethylamine (ⁱPr₂NEt), triethylamine (TEA), 4-methylmorpholine (NMM), imidazole, pyridine, and a collidine. Step (b) of Method 1 can be carried out in a solvent, such as an organic solvent, The organic solvent can be any solvent commonly known in the art for resin-based synthesis. Suitable organic solvents include, but are not limited to, CH₂Cl₂, THF, diethyl ether, MeOH, EtOH, acetone, acetonitrile, and the like. In some embodiments, the solvent is CH₂Cl₂. It will be appreciated that step (b) of Method 1 can be conducted at any temperature commonly used in solid-phase synthesis, such as room temperature, under cooling, or under warming conditions. The deprotection step (b) of Method 1 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the deprotection step (a) of Method 1, the resin-based compound of the formula C produced in step (b) can be washed with reagents commonly known in the art, such as DMF, IPA, CH₂Cl₂, and the like.

In step (c) of Method 1, the second deprotecting agent can be an acidic deprotecting agent, basic deprotecting agent, or other deprotecting agent depending on the identity of PG¹ and/or PG². Some embodiments, the second deprotecting agent can be an acidic deprotecting agent. Suitable acidic deprotecting agents include, but are not limited to, TFA, HCl, or H₃PO₄. In some embodiments, the second deprotecting agent in step (c) of Method 1 is selected from the group consisting of trifluoroacetic acid (TFA), acetic acid, trifluoroethanol, hexafluoroisopropanol, dichloromethane, and combinations thereof. In some embodiments, the second deprotecting is TFA, such as when PG² is Mtt. In some embodiments, the second deprotecting is a mixture of acetic acid and trifluoroethanol, such as when PG² is Mtt. In some embodiments, the second deprotecting agent can be a palladium deprotecting agent, such as when PG² is alloc. Suitable deprotecting agents and conditions for step (c) of Method 1 can be found in for example, Wuts, P. G. M., Greene, T. W., Greene, T. W., & John Wiley & Sons. (2006). Greene's protective groups in organic synthesis. Hoboken, N.J: Wiley-Interscience. an acidic deprotecting agent (TFA) is used to cleave Mtt. Other suitable acidic deprotecting agents include, but are not limited to, acetic acid, trifluoroethanol, dicloromethane, hexafluoroisopropanol, dichloromethane, and combinations thereof. Step (c) of Method 1 can be carried out in a solvent, such as an organic solvent. The organic solvent can be any solvent commonly known in the art for resin-based synthesis. Suitable organic solvents include, but are not limited to, CH₂Cl₂, THF, diethyl ether, MeOH, EtOH, acetone, acetonitrile, and the like. In some embodiments, the solvent is CH₂Cl₂.

In step (d), PG¹ on the PG¹-3-(2-napthyl)-L-alanine

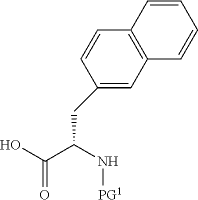

can be a protecting group, such as 9-Fluorenylmethyl-carbonyl (Fmoc) or 1-butylcarbonyl (Boc). See step (a) of Method 1 above regarding the choice of PG¹. In step (d) of Method 1, the polar aprotic solvent can be any polar aprotic solvent commonly known in the art. Suitable polar aprotic solvents include, but are not limited to, N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO). In some embodiments, the polar aprotic solvent is DMF. Suitable bases include, but are not limited to, triethylamine (TEA), 4-methylmorpholine (NMM), imidazole, pyridine, and collidine. In some embodiments, the base in step (d) of Method 1 is N,N-Diisopropylethylamine (ⁱPr₂NEt). The coupling agent can be any coupling agent commonly known in the art for resin-based synthesis. Suitable coupling agents include, but are not limited to, HATU, TBTU, HBTU, CDMT, HDMC, COMU, TATU, TOTT, T3P, DMTMM. In some embodiments, the coupling agent in step (d) of Method 1 is (PyBOP). It will be appreciated that step (d) of Method 1 can be conducted at any temperature commonly used in solid-phase synthesis, such as room temperature, under cooling, or under warming conditions. The coupling step (d) of Method 1 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the coupling step (d) of Method 1, the resin-based compound of the formula D produced in step (d) of Method 1 can be washed with reagents commonly known in the art, such as DMF, IPA, CH₂Cl₂, and the like.

In step (e) of Method 1, PG¹ on the resin-based compound of the formula D can be a protecting group, such as 9-Fluorenylmethyl-carbonyl (Fmoc) or t-butylcarbonyl (Boc), and PG² can be an orthogonal protecting group, such as 4-methyltrityl (Mtt) or monomethoxytrityl (MMt). The first deprotecting agent in step (e) of Method 1 can be an acidic or basic protecting agent, depending on the identity of PG¹ and/or PG². In particular, the first deprotecting agent can be a basic deprotecting agent, such as piperidine. morpholine, 1,8-diazabicyclo[5.4.0]undec-7-ene (DBU), diethylamine (DEA), dicyclohexamine, 4-methylpiperidine (4MP), Tris(2-aminoethyl)amine, pyridine, or a collidine. In particular, the first deprotecting agent can be an acidic deprotecting agent, such as TFA, HCl, or H₃PO₄. Suitable deprotecting agents and conditions for step (e) of Method 1 can be found in for example, Wuts, P. G. M., Greene, T. W., Greene, T. W., & John Wiley & Sons. (2006). Greene's protective groups in organic synthesis. Hoboken, N.J: Wiley-Interscience. The polar-aprotic solvent of step (e) of Method 1 can be any polar-aprotic solvent known in the art. Suitable polar-aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO), and the like. It will be appreciated that step (e) of Method 1 can be conducted at any temperature commonly used in solid-phase synthesis, such as room temperature, under cooling, or under warming conditions. The deprotection step (e) of Method 1 can be carried out for a period of time, such as from about 5 minutes to about 200 minutes, or about 10 minutes to about 100 minutes, or about 40 to about 60 minutes to about 45 minutes, or about 60 minutes, or about 90 minutes. Following the deprotection step (e) of Method 1, the resin-based compound of the formula D′ produced in step (e) of Method 1 can be washed with reagents commonly known in the art, such as DMF, IPA, CH₂Cl₂, and the like.

In step (f) of Method 1, PG¹ on the PG¹-tranexamic acid reagent

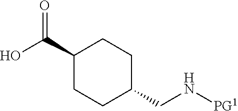

can be a protecting group, such as 9-Fluorenylmethyl-carbonyl (Fmoc) or 1-butylcarbonyl (Boc). See step (a) of Method 1 above regarding the choice of PG¹. In step (f) of Method 1, the polar aprotic solvent can be any polar aprotic solvent commonly known in the art. Suitable polar aprotic solvents include, but are not limited to, N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO). In some embodiments, the polar aprotic solvent is DMF. Suitable bases include, but are not limited to, triethylamine (TEA), 4-methylmorpholine (NMM), imidazole, pyridine, and collidine. In some embodiments, the base in step (f) of Method 1 is N,N-Diisopropylethylamine (ⁱPr₂NEt). The coupling agent can be any coupling agent commonly known in the art for resin-based synthesis. Suitable coupling agents include, but are not limited to, HATU, TBTU, HBTU, CDMT, HDMC, COMU, TATU, TOTT, T3P, DMTMM. In some embodiments, the coupling agent in step (f) of Method 1 is (PyBOP). It will be appreciated that step (f) of Method 1 can be conducted at any temperature commonly used in solid-phase synthesis, such as room temperature, under cooling, or under warming conditions. The coupling step (f) of Method 1 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the coupling step (f) of Method 1, the resin-based compound of the formula E produced in step (f) of Method 1 can be washed with reagents commonly known in the art, such as DMF, IPA, CH₂Cl₂, and the like.

In step (g) of Method 1, PG¹ on the resin-based compound of the formula E can be a protecting group, such as 9-Fluorenylmethyl-carbonyl (Fmoc) or t-butylcarbonyl (Boc), and PG² can be an orthogonal protecting group, such as 4-methyltrityl (Mtt) or monomethoxytrityl (MMt). The first deprotecting agent in step (g) of Method 1 can be an acidic or basic protecting agent, depending on the identity of PG¹ and/or PG². In particular, the first deprotecting agent can be a basic deprotecting agent, such as piperidine. morpholine, 1,8-diazabicyclo[5.4.0]undec-7-ene (DBU), diethylamine (DEA), dicyclohexamine, 4-methylpiperidine (4MP), Tris(2-aminoethyl)amine, pyridine, or a collidine. In particular, the first deprotecting agent can be an acidic deprotecting agent, such as TFA, HCl, or H₃PO₄. Suitable deprotecting agents and conditions for step (g) of Method 1 can be found in for example, Wuts, P. G. M., Greene, T. W., Greene, T. W., & John Wiley & Sons. (2006). Greene's protective groups in organic synthesis. Hoboken, N.J: Wiley-Interscience. The polar-aprotic solvent of step (g) of Method 1 can be any polar-aprotic solvent known in the art. Suitable polar-aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO), and the like. It will be appreciated that step (g) of Method 1 can be conducted at any temperature commonly used in solid-phase synthesis, such as room temperature, under cooling, or under warming conditions. The deprotection step (g) of Method 1 can be carried out for a period of time, such as from about 5 minutes to about 200 minutes, or about 10 minutes to about 100 minutes, or about 40 to about 60 minutes to about 45 minutes, or about 60 minutes, or about 90 minutes. Following the deprotection step (g) of Method 1, the resin-based compound of the formula E′ produced in step (g) of Method 1 can be washed with reagents commonly known in the art, such as DMF, IPA, CH₂Cl₂, and the like.

In step (h) of Method 1, the polar aprotic solvent can be any polar aprotic solvent commonly known in the art. Suitable polar aprotic solvents include, but are not limited to, N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO). In some embodiments, the polar aprotic solvent is DMF. Suitable bases include, but are not limited to, triethylamine (TEA), 4-methylmorpholine (NMM), imidazole, pyridine, and collidine. In some embodiments, the base in step (h) of Method 1 is N,N-Diisopropylethylamine (ⁱPr₂NEt). The coupling agent can be any coupling agent commonly known in the art for resin-based synthesis. Suitable coupling agents include, but are not limited to, HATU, TBTU, HBTU, CDMT, HDMC, COMU, TATU, TOTT, T3P, DMTMM. In some embodiments, the coupling agent in step (h) of Method 1 is (PyBOP). It will be appreciated that step (h) of Method 1 can be conducted at any temperature commonly used in solid-phase synthesis, such as room temperature, under cooling, or under warming conditions. The coupling step (h) of Method 1 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the coupling step (h) of Method 1, the resin-based compound of the formula F produced in step (h) of Method 1 can be washed with reagents commonly known in the art, such as DMF, IPA, CH₂Cl₂, and the like.

In step (i) of Method 1, the third deprotecting agent can be the same or different from the cleavage reagent. In some embodiments, the third deprotecting agent can be, for example, HF or TfOH, such as when R¹ is cyclohexyl and the cleavage reagent can be TFA. In some embodiments, such as when R¹ is tert-butyl, the third deprotecting agent and the cleavage reagent can both be TFA. In some embodiments, the third deprotecting agent and the cleavage reagent can both be a mixture of TFA, water, and triisopropylsilane. Other suitable deprotecting agents include, but are not limited to, a trifluoroacetic acid (TFA)/dichloromethane (DCM) mixture, triethylsaline (TES), indole, a phenol/anisole mixture, and thioanisole. Step (i) of Method 1 can be carried out in a solvent, such as an organic solvent. The organic solvent can be any solvent commonly known in the art for resin-based synthesis. Suitable organic solvents include, but are not limited to, CH₂Cl₂, THF, diethyl ether, MeOH, EtOH, acetone, acetonitrile, and the like. In some embodiments, the solvent is CH₂Cl₂.

In step (a) of Method 2, PG on the resin-based compound of the formula 1 can be a protecting group, and in particular can be an amine protecting group, such as 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), or 4-methyltrityl (Mtt). Also in step (a) of Method 2, PG¹ can be a protecting group, in particular an amine protecting group, such as 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), or 4-methyltrityl (Mtt) that is orthogonal to PG. In some embodiments, PG can be 9-fluorenylmethyl-carbonyl (Fmoc) or t-butylcarbonyl (Boc), and PG¹ can be an orthogonal protecting group, such as 4-methyltrityl (Mtt) or monomethoxytrityl (MMt).

The first deprotecting agent in step (a) of Method 2 can be a reagent or mixture of reagents capable of removing an amine protecting group, such as 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), or 4-methyltrityl (Mtt). In some embodiments, the first deprotecting agent of step (a) of Method 2 comprises a reagent or mixture of reagents capable of removing a 9-fluorenylmethyl-carbonyl. In some embodiments, the first deprotecting agent of step (a) of Method 2 can be an acidic or basic deprotecting agent, depending on the identity of PG and/or PG¹. In particular, the first deprotecting agent can be a basic deprotecting agent, such as piperidine. morpholine, 1,8-diazabicyclo[5.4.0]undec-7-ene (DBU), diethylamine (DEA), dicyclohexamine, 4-methylpiperidine (4MP), tris (2-aminoethyl)amine, pyridine, or a collidine. In particular, the first deprotecting agent can be an acidic deprotecting agent, such as TFA, HCl, or H₃PO₄. Suitable deprotecting agents and conditions for step (a) of Method 2 can be found in for example, Wuts, P. G. M., Greene, T. W., Greene, T. W., & John Wiley & Sons. (2006). Greene's protective groups in organic synthesis. Hoboken, N.J: Wiley-Interscience.

In some embodiments of step (a) of Method 2, the base used to remove the Fmoc group can be an amine base, such as piperidine. In some embodiments, the polar-aprotic solvent of step (a) of Method 2 can be any polar-aprotic solvent known in the art. Suitable polar-aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO), and the like. It will be appreciated that step (a) can be conducted at any temperature commonly used in solid-phase synthesis, such as room temperature, under cooling, or under warming conditions. The deprotection step (a) of Method 2 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the deprotection step (a) of Method 2, the resin-based compound of the formula B produced in step (a) of Method 2 can be washed with reagents commonly known in the art, such as DMF, IPA, CH₂Cl₂, and the like.

In step (b) of Method 2, PG² and/or PG³ on the compound of the formula 3 can each independently be carboxyl protecting groups, and LG is a leaving group, such as an active ester. Suitable active esters known in the art include, but are not limited to imidazolyl, 4-nitrophenoxy, phenoxy, 2,4,5-trichlorophenyl, NHS ester, benzotriazyl, and the like. In some embodiments, the LG in compound 3 is s selected from imidazolyl, 4-nitrophenoxy, and phenoxy. In some embodiments, the LG in compound 3 is a 4-nitrophenyl ester. It will be appreciated that the choice of PG² and/or PG³ will be orthogonal to the choice for PG and PG¹. In some embodiments, PG² and/or PG³ can be t-butyl. In step (b) of Method 2, the base can be any base commonly known in the art for urea formation reactions. Suitable bases include, but are not limited to, N,N-diisopropylethylamine (ⁱPr₂NEt), triethylamine (TEA), 4-methylmorpholine (NMM), imidazole, pyridine, and a collidine.

Step (b) of Method 2 can be carried out in a solvent, such as an organic solvent. The organic solvent can be any solvent commonly known in the art for resin-based synthesis. Suitable organic solvents include, but are not limited to, CH₂Cl₂, THF, diethyl ether, MeOH, EtOH, acetone, acetonitrile, and the like. In some embodiments, the solvent is CH₂Cl₂. It will be appreciated that step (b) of Method 2 can be conducted at any temperature commonly used in solid-phase synthesis, such as room temperature, under cooling, or under warming conditions. The deprotection step (b) of Method 2 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the step (b) of Method 2, the resin-based compound of the formula 4 produced in step (b) can be washed with reagents commonly known in the art, such as DMF, IPA, CH₂Cl₂, and the like.

In step (c) of Method 2, the second deprotecting agent can be a reagent or mixture of reagents capable of removing a protecting group selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt). In some embodiments, the second deprotecting agent can be a reagent or mixture of reagents capable of removing a 4-methyltrityl (Mtt). In some embodiments, the second deprotecting agent can be an acidic deprotecting agent, basic deprotecting agent, or other deprotecting agent depending on the identity of PG¹, PG², and/or PG³. In some embodiments, the second deprotecting agent can be an acidic deprotecting agent. Suitable acidic deprotecting agents include, but are not limited to, TFA, HCl, or H₃PO₄. In some embodiments, the second deprotecting agent in step (c) of Method 2 is selected from the group consisting of trifluoroacetic acid (TFA), acetic acid, trifluoroethanol, hexafluoroisopropanol, and combinations thereof. In some embodiments, the second deprotecting is TFA, such as when PG² is Mtt. In some embodiments, the second deprotecting in step (c) of Method 2 is a mixture of acetic acid and trifluoroethanol, such as when PG² is Mtt. Suitable deprotecting agents and conditions for step (c) of Method 2 can be found in for example, Wuts, P. G. M., Greene, T. W., Greene, T. W., & John Wiley & Sons. (2006). Greene's protective groups in organic synthesis. Hoboken, N.J: Wiley-Interscience. Step (c) of Method 2 can be carried out in a solvent, such as an organic solvent. The organic solvent can be any solvent commonly known in the art for resin-based synthesis. Suitable organic solvents include, but are not limited to, CH₂Cl₂, THF, diethyl ether, MeOH, EtOH, acetone, acetonitrile, and the like. In some embodiments, the solvent is CH₂Cl₂. Following the deprotection step (c) of Method 2, the resin-based compound of the formula 5 produced in step (c) of Method 2 can be washed with reagents commonly known in the art, such as DMF, IPA, CH₂Cl₂, and the like.

In step (d) of Method 2, PG⁴ can be a protecting group, in particular an amine protecting group, 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt). In some embodiments, PG⁴ can be 9-Fluorenylmethyl-carbonyl (Fmoc) or t-butylcarbonyl (Boc). See step (a) of Method 2 above regarding the choice of PG⁴. In step (d) of Method 2, the solvent can be a polar aprotic solvent. In some embodiments, the polar aprotic solvent can be any polar aprotic solvent commonly known in the art. Suitable polar aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO). In some embodiments, the polar aprotic solvent is DMF. Suitable bases for use in step (d) of Method 2 include, but are not limited to, N,N-diisopropylethylamine (ⁱPr₂NEt), triethylamine (TEA), 4-methylmorpholine (NMM), imidazole, pyridine, and collidine. In some embodiments, the base in step (d) is N,N-diisopropylethylamine (ⁱPr₂NEt).

The coupling agent can be any coupling agent commonly known in the art for resin-based synthesis. Suitable coupling agents for use in step (d) of Method 2 include, but are not limited to, benzotriazol-1-yloxy-tris (dimethylamino)-phosphonium hexafluorophosphate (BOP), benzotriazol-1-yl-oxytripyrrolidinophosphonium hexafluorophosphate (PyBOP), 7-aza-benzotriazol-1-yloxy-tripyrrolidino-phosphonium hexafluorophosphate (PyAOP), ethyl cyano (hydroxyimino) acetato-O₂)-tri-(1-pyrrolidinyl)-phosphonium hexafluorophosphate (PyOxim), bromo-tripyrrolidino-phosphonium hexafluorophosphate (PyBrOP), 3-(diethoxy-phosphoryloxy)-1,2,3-benzo[d]triazin-4 (3H)-one (DEPBT), 1-[bis(dimethylamino)methylene]-1H-1,2,3-triazolo[4,5-b]pyridinium 3-oxid hexafluorophosphate (HATU), 2-(1H-benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-(6-chloro-1H-benzotriazol-1-yl)-N,N,N′,N′-tetramethylaminium hexafluorophosphate (HCTU), 2-chloro-4,6-dimethoxy-1,3,5-triazine (CDMT), N-[(5-chloro-3-oxido-1H-benzotriazol-1-yl)-4-morpholinylmethylene]-N-methylmethanaminium hexafluorophosphate (HDMC), 1-cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium tetrafluoroborate (TATU), N,N,N,N-tetramethyl-S-(1-oxido-2-pyridyl)thiouronium tetrafluoroborate (TOTT), tetramethylfluoroformamidinium hexafluorophosphate (TFFH), N-ethoxycarbonyl-2-ethoxy-1,2-dihydroquinoline (EEDQ), 1-propanephosphonic anhydride (T3P), 4-(4,6-dimethoxy-1,3,5-triazin-2-yl)-4-methyl-morpholinium chloride (DMTMM), dicyclohexylcarbodiimide (DCC), diisopropylcarbodiimide (DIC), (N-(3-dimethylaminopropyl)-N′-ethylcarbodiimide (EDAC), or 1,1′-carbonyldimidizole (CDI). In some embodiments, the coupling agent of step (d) of Method 2 can be HATU, TBTU, HBTU, CDMT, HDMC, COMU, TATU, TOTT, T3P, or DMTMM. In some embodiments, the coupling agent in step (d) of Method 2 is (PyBOP).

It will be appreciated that step (d) of Method 2 can be conducted in the presence of an additive. Suitable additive include, but are not limited to, 1-hydroxybenzotriazole (HOBt), 1-hydroxybenzotriazole-6-sulfonamidomethyl resin HCl (HOBt-6-sulfonamidomethyl resin HCl), hydroxy-3,4-dihydro-4-oxo-1,2,3-benzotriazine (HOOBt), N-hydroxysuccinimide (NHS), 1-hydroxy-7-aza-1H-benzotriazole (HOAt), ethyl 2-cyano-2-(hydroximino)acetate, or 4-(N,N-dimethylamino)pyridine (DMAP). It will be appreciated that step (d) of Method 2 can be conducted at any temperature commonly used in solid-phase synthesis, such as room temperature, under cooling, or under warming conditions. The coupling step (d) of Method 2 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the coupling step (d) of Method 2, the resin-based compound of the formula 7 produced in step (d) of Method 2 can be washed with reagents commonly known in the art, such as DMF, IPA, CH₂Cl₂, and the like.

In step (e) of Method 2, PG⁴ on the resin-based compound of the formula 7 can be an amine protecting group, such as 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt). The third deprotecting agent in step (e) of Method 2 can be a reagent or mixture of reagents capable of removing a protecting group selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt). In some embodiments, PG⁴ can be 9-Fluorenylmethyl-carbonyl (Fmoc). In some embodiments, the third deprotecting agent can be the third deprotecting agent of step (e) of Method 2 comprises a reagent or mixture of reagents capable of removing a 9-fluorenylmethyl-carbonyl. In some embodiments, the third deprotecting agent can be an acidic or basic protecting agent. In some embodiments, the third deprotecting agent can be a basic deprotecting agent, such as piperidine. morpholine, 1,8-diazabicyclo[5.4.0]undec-7-ene (DBU), diethylamine (DEA), dicyclohexamine, 4-methylpiperidine (4MP), Tris(2-aminoethyl)amine, pyridine, or a collidine. In some embodiments, the third deprotecting agent can be an acidic deprotecting agent, such as TFA, HCl, or H₃PO₄. Suitable deprotecting agents and conditions for step (e) of Method 2 can be found in for example, Wuts, P. G. M., Greene, T. W., Greene, T. W., & John Wiley & Sons. (2006). Greene's protective groups in organic synthesis. Hoboken, N.J: Wiley-Interscience.

In some embodiments, step (e) of Method 2 can be carried out in a solvent, such as a polar-aprotic solvent. The polar-aprotic solvent of step (e) of Method 2 can be any polar-aprotic solvent known in the art. Suitable polar-aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO), and the like. It will be appreciated that step (e) of Method 2 can be conducted at any temperature commonly used in solid-phase synthesis, such as room temperature, under cooling, or under warming conditions. The deprotection step (e) of Method 2 can be carried out for a period of time, such as from about 5 minutes to about 200 minutes, or about 10 minutes to about 100 minutes, or about 40 to about 60 minutes to about 45 minutes, or about 60 minutes, or about 90 minutes. Following the deprotection step (e) of Method 2, the resin-based compound of the formula 9 produced in step (e) of Method 2 can be washed with reagents commonly known in the art, such as DMF, IPA, CH₂Cl₂, and the like.

In step (f) of Method 2, PG⁵ can be a carboxyl protecting group. In some embodiments, PG⁵ can be t-butyl. See step (b) of Method 2 above regarding the choice of PG⁵. In step (f) of Method 2, the solvent can be a polar aprotic solvent. In some embodiments, the polar aprotic solvent can be any polar aprotic solvent commonly known in the art. Suitable polar aprotic solvents include, but are not limited to, dimethylformamide (DMF), N-methyl-2-pyrrolidone (NMP), dichloromethane (DCM), a dichloromethane/dimethylformamide mixture, acetonitrile (ACN), an acetonitrile/dimethylformamide mixture, and dimethylsulfoxide (DMSO). In some embodiments, the polar aprotic solvent is DMF. Suitable bases for use in step (f) include, but are not limited to, N,N-diisopropylethylamine (ⁱPr₂NEt), triethylamine (TEA), 4-methylmorpholine (NMM), imidazole, pyridine, and collidine. In some embodiments, the base in step (f) of Method 2 is N,N-Diisopropylethylamine (ⁱPr₂NEt). The coupling agent of step (f) of Method 2 can be any coupling agent commonly known in the art for resin-based synthesis. Suitable coupling agents for use in step (f) of Method 2 include, but are not limited to, benzotriazol-1-yloxy-tris (dimethylamino)-phosphonium hexafluorophosphate (BOP), benzotriazol-1-yl-oxytripyrrolidinophosphonium hexafluorophosphate (PyBOP), 7-aza-benzotriazol-1-yloxy-tripyrrolidino-phosphonium hexafluorophosphate (PyAOP), ethyl cyano (hydroxyimino) acetato-O₂)-tri-(1-pyrrolidinyl)-phosphonium hexafluorophosphate (PyOxim), bromo-tripyrrolidino-phosphonium hexa-fluorophosphate (PyBrOP), 3-(diethoxy-phosphoryloxy)-1,2,3-benzo[d]triazin-4 (3H)-one (DEPBT), 1-[bis(dimethylamino)methylene]-1H-1,2,3-triazolo[4,5-b]pyridinium 3-oxid hexafluorophosphate (HATU), 2-(1H-benzotriazole-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-(6-chloro-1H-benzotriazol-1-yl)-N,N,N′,N′-tetramethylaminium hexafluorophosphate (HCTU), 2-chloro-4,6-dimethoxy-1,3,5-triazine (CDMT), N-[(5-chloro-3-oxido-1H-benzotriazol-1-yl)-4-morpholinylmethylene]-N-methylmethanaminium hexafluorophosphate (HDMC), 1-cyano-2-ethoxy-2-oxoethylidenaminooxy)dimethylamino-morpholino-carbenium hexafluorophosphate (COMU), dimethylamino (triazolo[4,5-b]pyridin-3-yloxy)methylidene]-dimethylazanium tetrafluoroborate (TATU), N,N,N′,N′-tetramethyl-S-(1-oxido-2-pyridyl)thiouronium tetrafluoroborate (TOTT), tetramethylfluoroformamidinium hexafluorophosphate (TFFH), N-ethoxycarbonyl-2-ethoxy-1,2-dihydroquinoline (EEDQ), 1-propanephosphonic anhydride (T3P), 4-(4,6-dimethoxy-1,3,5-triazin-2-yl)-4-methyl-morpholinium chloride (DMTMM), dicyclohexylcarbodiimide (DCC), diisopropylcarbodiimide (DIC), (N-(3-dimethylaminopropyl)-N′-ethylcarbodiimide (EDAC), and 1,1′-carbonyldimidizole (CDI). In some embodiments, the coupling agent of step (f) of Method 2 can be HATU, TBTU, HBTU, CDMT, HDMC, COMU, TATU, TOTT, T3P, or DMTMM. In some embodiments, the coupling agent in step (f) of Method 2 is (PyBOP).

It will be appreciated that step (f) of Method 2 can be conducted in the presence of an additive. Suitable additive include, but are not limited to, 1-hydroxybenzotriazole (HOBt), 1-hydroxybenzotriazole-6-sulfonamidomethyl resin HCl (HOBt-6-sulfonamidomethyl resin HCl), hydroxy-3,4-dihydro-4-oxo-1,2,3-benzotriazine (HOOBt), N-hydroxysuccinimide (NHS), 1-hydroxy-7-aza-1H-benzotriazole (HOAt), ethyl 2-cyano-2-(hydroximino)acetate, or 4-(N,N-dimethylamino)pyridine (DMAP). It will be appreciated that step (f) of Method 2 can be conducted at any temperature commonly used in solid-phase synthesis, such as room temperature, under cooling, or under warming conditions. The coupling step (f) of Method 2 can be carried out for a period of time, such as from about 5 minutes to about 200 minutes, or about 10 minutes to about 100 minutes, or about 40 to about 60 minutes to about 15 minutes, or about 30 minutes, or about 90 minutes. Following the coupling step (f) of Method 2, the resin-based compound of the formula 10 produced in step (f) of Method 2 can be washed with reagents commonly known in the art, such as DMF, IPA, CH₂Cl₂, and the like.

In step (g) of Method 2, PG², PG³, and/or PG⁵ on the resin-based compound of the formula 10 can each be a protecting group, such as a carboxyl protecting group. In some embodiments, the third deprotecting agent of step (g) of Method 2 can be a reagent or mixture of reagents capable of removing a protecting group selected from the group consisting of t-butyl, benzyl, 2-chlorotrityl (2-Cl-Trt), 2,4-dimethoxybenzyl (Dmb), allyl, 1,1-dimethylallyl (Dma), and p-nitrobenzyl (pNB). In some embodiments, each of PG², PG³, and PG⁵ is a t-butyl group. The third deprotecting agent in step (g) of Method 2 can be an acidic or basic protecting agent, depending on the identity of PG², PG³, and/or PG⁵. In some embodiments, the third deprotecting agent can be an acidic deprotecting agent, such as TFA, HCl, or H₃PO₄. In some embodiments, the third deprotecting agent can be selected from the group consisting of trifluoroacetic acid (TFA), hydrofluoric acid (HF), triflic acid (TfOH), a trifluoroacetic acid (TFA)/water/triisopropylsilane mixture, a trifluoroacetic acid (TFA)/dichloromethane (DCM) mixture, triethylsaline (TES), indole, a phenol/anisole mixture, and thioanisole. Suitable deprotecting agents and conditions for step (g) can be found in for example, Wuts, P. G. M., Greene, T. W., Greene, T. W., & John Wiley & Sons. (2006). Greene's protective groups in organic synthesis. Hoboken, N.J: Wiley-Interscience. In some embodiments, the third deprotecting agent of step (g) of Method 2 is a trifluoroacetic acid (TFA)/water/triisopropylsilane mixture. In some embodiments, the cleavage reagent of step (g) of Method 2 is a trifluoroacetic acid (TFA)/water/triisopropylsilane mixture.

It will be appreciated that the organic solvent of step (g) of Method 2 can be any organic solvent known in the art. Suitable organic solvents include, but are not limited to, CH₂Cl₂, THE, diethyl ether, MeOH, EtOH, acetone, acetonitrile, and the like. It will be appreciated that step (g) of Method 2 can be conducted at any temperature commonly used in solid-phase synthesis, such as room temperature, under cooling, or under warming conditions. The deprotection step (g) of Method 2 can be carried out for a period of time, such as from about 5 minutes to about 200 minutes, or about 10 minutes to about 100 minutes, or about 40 to about 60 minutes to about 45 minutes, or about 60 minutes, or about 90 minutes.

### Method 2, Specific Embodiment

PSMA 617 (I) can be prepared using solid phase peptide synthesis technology as shown in the following scheme.

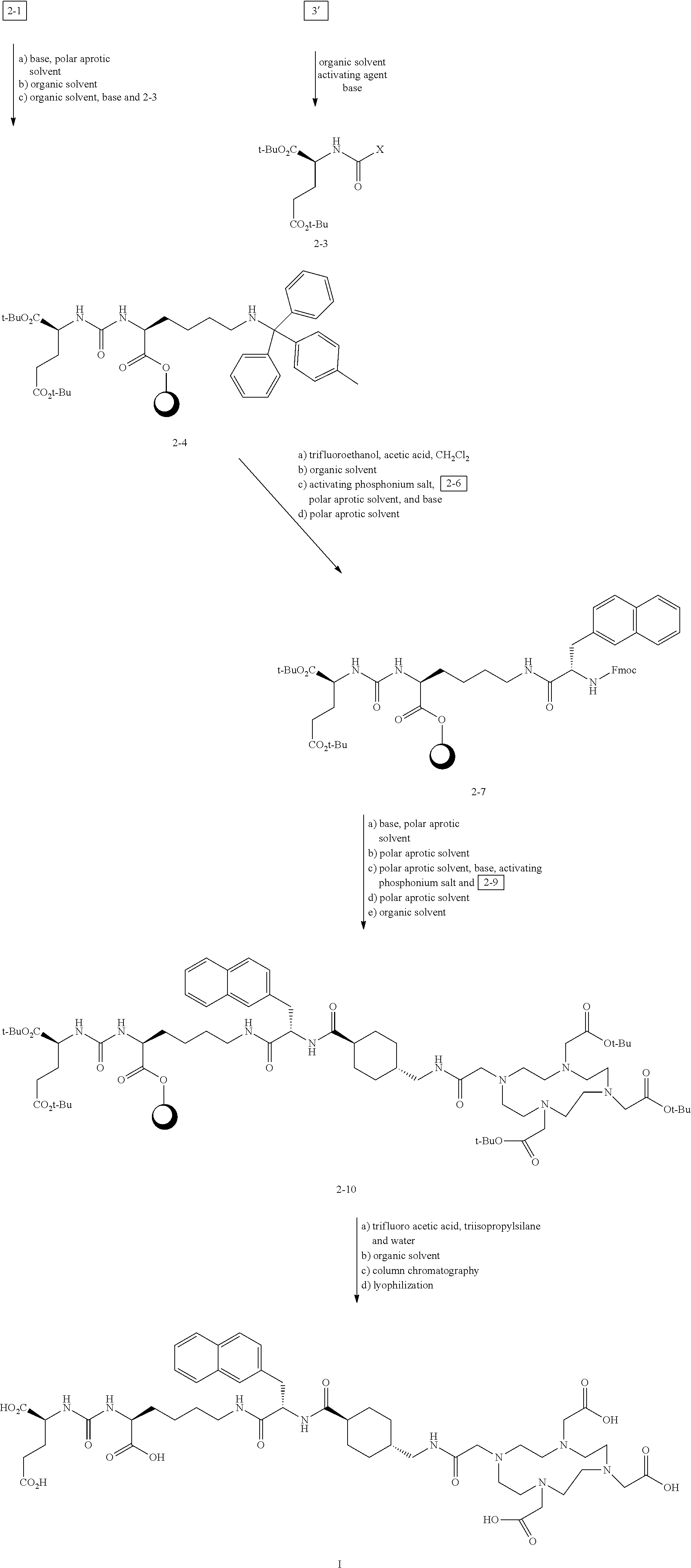

The following reagents and intermediates can be used.

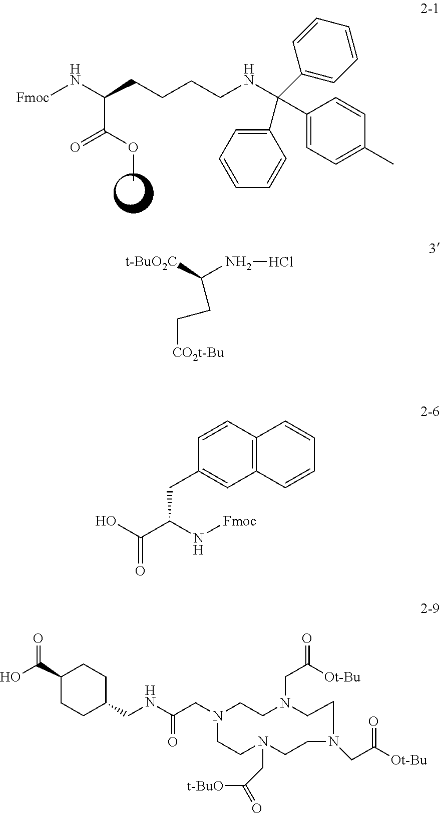

Three chemical transformations are performed. The Fmoc protecting group is removed from Fmoc-Lys (Mtt)-Wang Resin (2-1) by mixing the resin with a base in a polar aprotic solvent, and the resin is then washed with solvent. In an organic solvent, L-glutamic acid di-tert-butyl ester hydrochloride (3′) is converted into an activated urea or carbamate (2-3) using, for example, a base and carbonyldiimidazole, p-nitrophenyl chloroformate, phenyl chloroformate, and the like. The deprotected compound 2-2 is mixed with the solution of 2-3. When the transformation to 2-4 is complete, the resin is washed with an organic solvent.

The methyl trityl protecting group is removed from 2-4 by mixing the resin with a mixture 2,2,2-trifluoroethanol, acetic acid, and dichloromethane. Upon removal of the trityl group, the resin is washed with a polar aprotic solvent. The deprotected compound 2-5 is mixed with (2S)-2-(9-fluorenylmethyloxycarbonylamino)-3-naphthalen-2-yl-propionic acid (2-6), a base, and an activating agent, such as but not limited to a phosphonium salt activating agent in a polar aprotic solvent. After conversion into 2-7, the resin is washed with a polar aprotic solvent.

The Fmoc protecting group is removed from 2-7 by mixing the resin with a base in polar aprotic solvent, and washing the resin with solvent. The deprotected compound 2-8 is mixed with 2-9, a base, and an activating agent, such as but not limited to a phosphonium salt activating agent in a polar aprotic solvent. After conversion into 2-10, the resin is washed with a polar aprotic solvent and organic solvent.

The tert-butyl groups are removed and the resin is cleaved and by mixing with a mixture of trifluoroacetic acid, triisopropylsilane, and water. The liquors are separated from the cleaved resin, added to an organic solvent, and filtered. The crude PSMA 617 is purified by column chromatography, and the material is de-salted using a C18 column and an acetonitrile/water gradient eluent. The acetonitrile is evaporated or distilled from the aqueous solution under vacuum, and PSMA 617 (I) is isolated from the solution by lyophilization.

### Examples

The examples and preparations provided below further illustrate and exemplify particular aspects of embodiments of the disclosure. It is to be understood that the scope of the present disclosure is not limited in any way by the scope of the following examples.

### Abbreviations

The examples described herein use materials, including but not limited to, those described by the following abbreviations known to those skilled in the art:

| | |
|---|---|
| PG | Protecting group |
| LG | Leaving Group |
| IPA | Isopropyl alcohol |
| DBU | 1,8-Diazabicyclo[5.4.0]undec-7-ene |
| DEA | diethylamine |
| 4MP | 4-methylpiperidine |
| DMF | dimethylformamide |
| NMP | N-methyl-2-pyrrolidone |
| DCM | dichloromethane |
| ACN | acetonitrile |
| DMSO | dimethylsulfoxide |
| ⁱPr₂NEt | N,N-Diisopropylethylamine |
| TEA | triethylamine |
| NMM | 4-methylmorpholine |
| TFA | trifluoroacetic acid |
| PyBOP | benzotriazol-1-yl-oxytripyrrolidinophosphonium |
| | hexafluorophosphate |
| HATU | 1-[Bis(dimethylamino)methylene]-1H-1,2,3- |
| | triazolo[4,5-b]pyridinium 3-oxid hexafluorophosphate |
| TBTU | 2-(1H-Benzotriazole-1-yl)-1,1,3,3- |
| | tetramethyluronium tetrafluoroborate |
| HBTU | 2-(1H-benzotriazol-1-yl)-1,1,3,3- |
| | tetramethyluronium hexafluorophosphate |
| HDMC | N-[(5-Chloro-3-oxido-1H-benzotriazol-1-yl)-4- |
| | morpholinylmethylene]-N-methylmethanaminium |
| | hexafluorophosphate |
| COMU | 1-Cyano-2-ethoxy-2- |
| | oxoethylidenaminooxy)dimethylamino-morpholino- |
| | carbenium hexafluorophosphate |
| TATU | dimethylamino(triazolo[4,5- |
| | b]pyridin-3-yloxy)methylidene]- |
| | dimethylazanium; tetrafluoroborate |
| EEDQ | N-Ethoxycarbonyl-2-ethoxy-1,2-dihydroquinoline |
| T3P | 1-Propanephosphonic anhydride |
| DMTMM | 4-(4,6-dimethoxy-1,3,5-triazin-2- |
| | yl)-4-methyl-morpholinium chloride |
| TES | triethylsaline |
| Fmoc | 9-Fluorenylmethyl-carbonyl |
| Boc | t-butylcarbonyl |
| MMt | monomethoxytrityl |
| Mtt | 4-methyltrityl |
| g | grams |
| eq or equiv | equivalents |
| mmol | millimoles |
| mol | moles |
| mL | milliliters |
| L | liters |
| min or mins | minute or minutes |
| hrs, hr or h | hour or hours |
| M | molar |
| SPPS | Solid phase peptide synthesis |
| TfOH | Triflic acid |
| HF | Hydrofluoric acid |

### Example 1a: Preparation of AB2

Compound AB2 was prepared according to the following synthetic scheme:

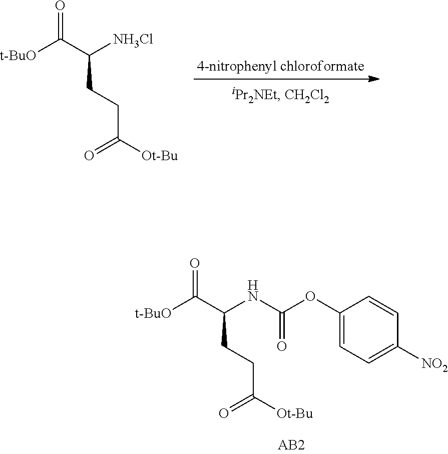

403 mg of 4-nitrophenyl chloroformate (2.0 mmol, 0.95 equiv) was added to a stirred solution of 621 mg of L-Glutamic acid di-tert-butyl ester hydrochloride (2.1 mmol, 1.0 equiv) in 21 mL of dichloromethane (CH₂Cl₂) in a round bottom flask under argon. The solution was cooled to 0° C., before a solution of 0.730 mL of diisopropylethylamine (ⁱPr₂NEt) (4.2 mmol, 2.0 equiv) in 5 mL of CH₂Cl₂ was added dropwise with a pressure-equalizing addition funnel. After the complete addition of ‘Pr₂NEt, the reaction mixture stirred for 30 min, warmed to room temperature, and stirred for an additional 30 min. The reaction mixture was then evaporated to yield the crude product as an oily residue. The residue was used without further purification and held under high vacuum until desired.

### Example 1b: Alternative Preparation of AB2

A reaction vessel was charged with 0.891 g of 4-nitrophenyl chloroformate (4.42 mmol) and 15 mL of dichloromethane (CH₂Cl₂), and was purged with argon. The reaction vessel was fitted with a temperature probe, and the solution was cooled to ≤−10° C. using an IPA/ice bath. In a separate reaction flask was dissolved 1.44 g of L-glutamic acid di-tert-butyl ester hydrochloride (4.87 mmol) in 15 mL of dichloromethane (CH₂Cl₂), followed by the addition of 1.54 mL of diisopropylethylamine (ⁱPr₂NEt) (8.84 mmol). The L-glutamic acid di-tert-butyl ester hydrochloride solution dropwise to the cooled 4-nitrophenyl chloroformate solution, while maintaining a temperature of −11° C. to −5° C. The ice bath was removed after the addition was completed, and the reaction mixture was stirred at room temperature for 4 hours, at which time the reaction was shown to be complete using UPLC/MS. The reaction mixture was used without further purification.

### Example 2: Preparation of PSMA-617

The compound of formula I was prepared by SPPS starting from Fmoc-Lys (N-4-methoxytrityl)-2-chlorotrityl-Resin. The compound of the formula I was prepared according to the following synthetic scheme:

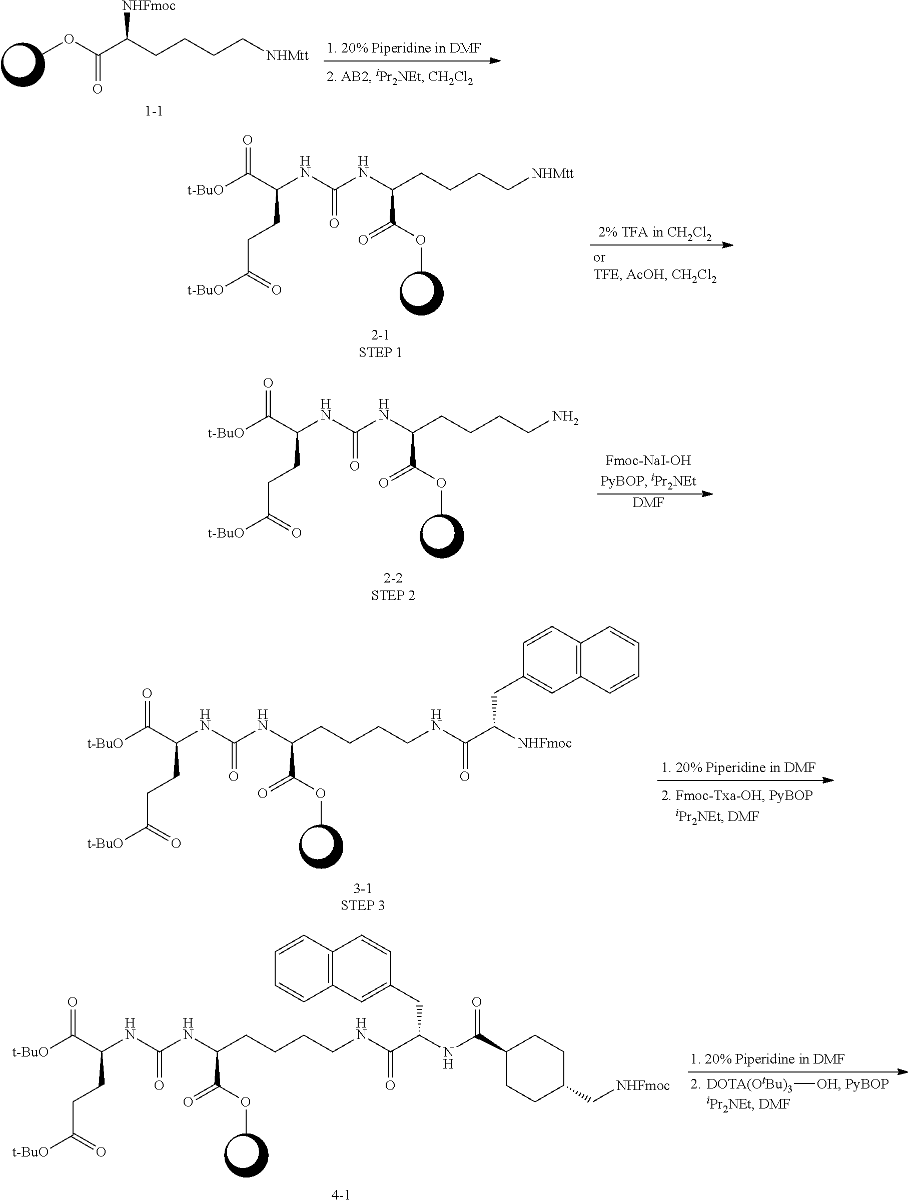

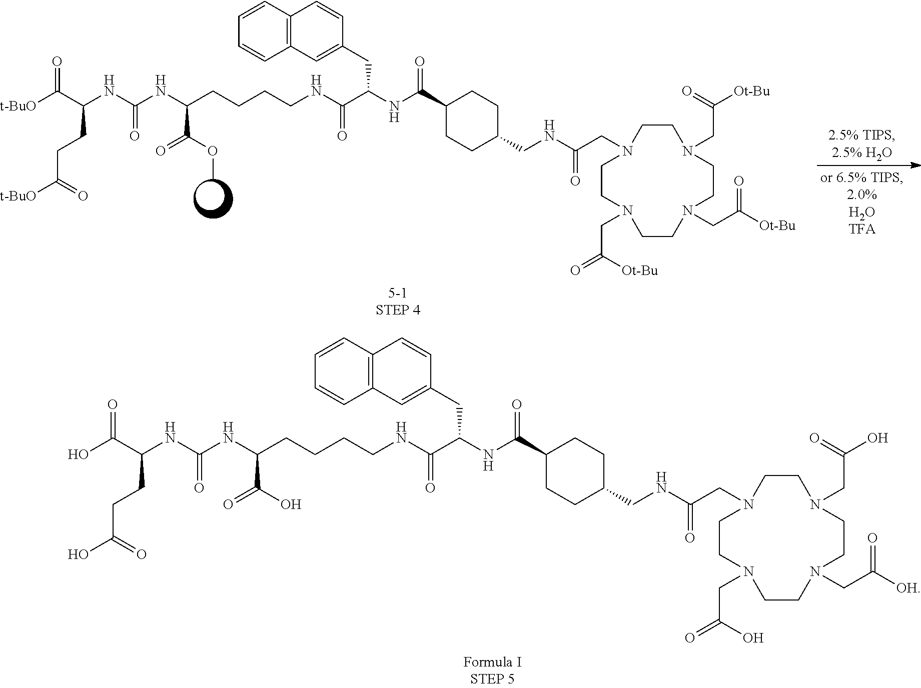 [SMILES: COC(=O)[C@H](CCCCNC(=O)[C@H](Cc1ccc2ccccc2c1)NC(=O)[C@H]1CC[C@H](CNC(=O)CN2CCN(CC(=O)OC(C)(C)C)CCN(CC(=O)OC(C)(C)C)CCN(CC(=O)OC(C)(C)C)CC2)CC1)NC(=O)N[C@@H](CCC(=O)OC(C)(C)C)C(=O)OC(C)(C)C ; O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

The following Tables I and 2 are summaries of the synthetic scheme described above as carried out according to the methods described herein:

TABLE 1
Reaction steps for synthetic scheme of PSMA-617.
Table 1: Solid phase reaction steps:

| | | | MW | | |
|---|---|---|---|---|---|
| | | | (g/ | | |
| | mmol. | equiv. | mol) | amount | |
| Fmoc-Lys(N-4-methoxytrityl)- | 0.5 | — | — | 1.47 | g |
| 2-chlorotrityl-Resin (loading | | | | | |
| 0.34 mmol/g.) | | | | | |

Fmoc Deprotection - 20% Piperidine in DMF (3X)
Urea Formation:

| | | | | | |
|---|---|---|---|---|---|
| AB2 | 1 | 2 | 424 | ½ | crude |
| ⁱPr₂NEt | 2 | 4 | 129 | 0.258 | mL |

Mtt Deprotection - 2% TFA in CH₂Cl₂ (6X)
Fmoc-3-(2-naphthyl)-L-alanine (Fmoc-2-Nal-OH) Coupling:

| | | | | | |
|---|---|---|---|---|---|
| Fmoc-Nal-OH | 1 | 2 | 438 | 438 | mg |
| ⁱPr₂NEt | 2 | 4 | 129 | 0.258 | mL |
| PyBOP | 1 | 2 | 520 | 520 | mg |

Fmoc Deprotection - 20% Piperidine in DMF (3X)
Fmoc-tranexamic acid (Fmoc-Txa-OH) Coupling:

| | | | | | |
|---|---|---|---|---|---|
| Fmoc-Txa-OH | 1 | 2 | 379 | 379 | mg |
| ⁱPr₂NEt | 2 | 4 | 129 | 0.258 | mL |
| PyBOP | 1 | 2 | 520 | 520 | mg |

Fmoc Deprotection - 20% Piperidine in DMF (3X)
Tri-tert-butyl 1,4,7,10-tetraazacyclododecane-1,4,7,10-
tetraacetate {DOTA(O^{t}Bu)₃—OH} Coupling:

| | | | | | |
|---|---|---|---|---|---|
| DOTA(O^{t}Bu)₃—OH | 1 | 1.25 | 573 | 716 | mg |
| ⁱPr₂NEt | 2 | 4 | 129 | 0.258 | mL |
| PyBOP | 1 | 2 | 520 | 520 | mg |

Cleavage - 2.5% TIPS, 2.5% H₂O, TFA

TABLE 2
Alternative solid phase reaction steps:

| | mmol. | equiv. | MW (g/mol) | amount | |
|---|---|---|---|---|---|
| Fmoc-Lys(N-4-methoxytrityl)-2-chlorotrityl- | 1.08 | 1 | — | 2.00 | g |
| Resin (loading 0.34 mmol/g.) | | | | | |

Pretreatment Step
Alternative Fmoc Deprotection - 20% Piperidine in DMF (3X)
Alternative Urea Formation:

| | | | | | |
|---|---|---|---|---|---|
| ⁱPr₂NEt | 1.08 | 1 | 129 | 0.188 | mL |

Alternative Mtt Deprotection - CH₂Cl₂/TFE/AcOH
Fmoc-3-(2-naphthyl)-L-alanine (Fmoc-2-Nal-OH) Coupling using Alternative Amino Coupling:

| | | | | | |
|---|---|---|---|---|---|
| Fmoc-Nal-OH | 2.16 | 2 | 438 | 945 | mg |
| ⁱPr₂NEt | 4.32 | 4 | 129 | 0.752 | mL |
| PyBOP | 2.16 | 2 | 520 | 1.12 | g |

Alternative Fmoc Deprotection - 20% Piperidine in DMF (3X)
Fmoc-tranexamic acid (Fmoc-Txa-OH) Coupling using Alternative Amino Coupling:

| | | | | | |
|---|---|---|---|---|---|
| Fmoc-Txa-OH | 2.16 | 2 | 379 | 819 | mg |
| ⁱPr₂NEt | 4.32 | 4 | 129 | 0.752 | mL |
| PyBOP | 2.16 | 2 | 520 | 1.12 | g |

Alternative Fmoc Deprotection - 20% Piperidine in DMF (3X)
Tri-tert-butyl 1,4,7,10-tetraazacyclododecane-1,4,7,10-tetraacetate {DOTA(O^{t}Bu)₃—OH}
Coupling using Alternative Amino Coupling:

| | | | | | |
|---|---|---|---|---|---|
| DOTA(O^{t}Bu)₃—OH | 2.16 | 2 | 573 | 1.24 | g |
| ⁱPr₂NEt | 4.32 | 4 | 129 | 0.752 | mL |
| PyBOP | 2.16 | 2 | 520 | 1.12 | g |

Alternative resin Cleavage - 6.5% TIPS, 2% H₂O in TFA
Purification Procedure 2

### Pretreatment Step:

Prior to use in the synthesis method, a peptide synthesis vessel was charged with Fmoc-Lys (Mtt)-Wang resin (resin loading=0.54 mmol/g) 2.00 g (1.08 mmol). The resin was washed with 20 mL of dimethylformamide (DMF) for 5 minutes, and then drain the liquors from the vessel. The washing step was repeated one additional time.

### Step 1: Urea Formation

In a peptide synthesis vessel was added the lysin resin. A solution of 20% piperidine in DMF (˜20 mL) for Fmoc deprotection was added. Argon was bubbled through the solution for 15 min and then drained. 20% piperidine in DMF (˜20 mL) was added and bubbling continued for 5 min before draining (repeated 2X). The resin was washed with DMF (˜20 mL X 3) followed by IPA (˜20 mL X 3) and finally with CH₂Cl₂ (˜20 mL X 3). Crude residue of AB2 was dissolved in 60 mL of CH₂Cl₂, and half the volume was added to the deprotected Lysine resin followed by ‘Pr₂NEt. Argon was bubbled through the solution for 45 min and then drained. The remaining crude AB2 solution followed by ‘Pr₂NEt was added to the resin, and argon was bubbled through the solution for an additional 30 min. The reaction vessel was then drained and the resin was washed with CH₂Cl₂ (˜20 mL X 3) followed by IPA (˜20 mL X 3) and again with CH₂Cl₂ (˜20 mL X 3).

### Alternative Urea Formation:

In a peptide synthesis vessel was added the lysin resin. The AB2 reaction mixture from Example 1b was added to the resin along with 0.188 mL ‘Pr₂NEt (1.08 mmoles). The reaction mixture was agitated overnight (approximately 18 hours). The resin was washed with CH₂Cl₂ (˜20 mL X 3).

### Step 2: Mtt Deprotection

2% TFA in CH₂Cl₂ (˜20 mL) was added and argon was bubbled through the solution for 5 min. Small amount of CH₂Cl₂ was added to the reaction vessel to maintain the same amount volume if bubbling vigorously. The yellow solution was then drained and repeated five times. The resin was washed with fresh CH₂Cl₂ until the filtrate remained clear. A 2% solution of TFA in CH₂Cl₂ was added once more. If the solution remained clear, the reaction mixture was drained, and the next coupling step was performed. If the solution turned yellow the resin was washed with fresh CH₂Cl₂ until clear and the process was repeated until a clear reaction solution was achieved. The resin was washed with DMF (˜20 mL X 3).

### Alternative Mtt Deprotection

A solution of 112 mL CH₂Cl₂, 32 mL 2,2,2-trifluoroethanol (TFE), and 16 mL acetic acid (AcOH) was prepared. 20 mL of the AcOH-TFE solution was added to the resin in a peptide synthesis vessel. The mixture was agitated for about 1 hour, and the liquors were drained from the peptide synthesis vessel. The procedure was repeated one additional time. 20 mL of the AcOH-TFE solution was added to the resin in a peptide synthesis vessel, and the mixture was agitated overnight. The liquors were drained the from the peptide synthesis vessel. 20 mL of the AcOH-TFE solution was added to the resin in a peptide synthesis vessel, and the mixture was agitated for about 1 hour. The liquors were drained from the peptide synthesis vessel. The procedure was repeated one additional time. The resin was washed with 3× CH₂Cl₂, 3×2-propanol (IPA), and 3× DMF.

### Step 3: Amino Coupling

An amino acid solution in DMF (˜20 mL), ‘Pr₂NEt, and PyBOP were added to a peptide synthesis vessel. Argon was bubbled through the solution for 1 h and then drained. The resin was washed with DMF (˜20 mL X 3) followed by IPA (˜20 mL X 3) and with DMF (˜20 mL X 3) again.

### Alternative Amino Coupling

An amino acid solution in DMF (˜20 mL), PyBOP in DMF (˜20 mL), and ‘Pr₂NEt were added to a peptide synthesis vessel containing the resin. The mixture was agitated for about 4 hours and then the liquors were drained from the peptide synthesis vessel. The resin was washed with DMF (˜20 mL X 3) followed by IPA (˜20 mL X 3).

### Step 4: Fmoc Deprotection

The deprotection step was performed before each amino acid coupling steps (besides the Mtt deprotection which used 2% TFA in CH₂Cl₂). A solution of 20% piperidine in DMF (˜20 mL) for Fmoc deprotection was added. Argon was bubbled through the solution for 15 min and then drained. 20% piperidine in DMF (˜20 mL) was added and bubbling continued for 5 min before draining (2×). The resin was washed with DMF (˜20 mL X 3) followed by IPA (˜20 mL X 3) and with DMF again (˜20 mL X 3).

### Alternative FMOC Deprotection

The alternative deprotection step was performed before each amino acid coupling steps (besides the Mtt deprotection). To the resin was added a solution of 20% piperidine in DMF (˜20 mL). The reaction was agitated for about 10 minutes. The liquors were drained from the reaction vessel, and the process repeated two additional times. The resin was washed with DMF (˜20 mL X 3) followed by IPA (˜20 mL X 3). In some instances, the resin was further washed with DMF (˜20 mL X 3) or alternatively with CH₂Cl₂ (˜20 mL X 3).

### Step 5: Resin Cleavage

The resin was washed with CH₂Cl₂ (˜20 mL X 3) and drained. 25 mL of cleavage reagent (95% TFA, 2.5% H₂O, 2.5% Triisopropylsilane) was added to the peptide synthesis vessel and Argon was bubbled for 1 h, drained, and repeated twice with fresh cleavage reagent (10 mL for 15 min). The filtrate was stirred overnight under argon at room temperature. The reaction mixture was concentrated under reduced pressure until 10 ml remained. The product was triturated in 40 mL of diethyl ether and centrifuged. The solution was decanted from the resulting pellet. The previous step was repeated twice by resuspending the pellet in 50 mL of diethyl ether and centrifuging. The pellet was dried over a stream of argon and then high vacuum.

### Alternative Resin Cleavage

A mixture of 42 mL trifluoroacetic acid (TFA), 0.90 mL water, and 3.0 mL triisopropylsilane (TIPS) was prepared, and added to the resin in a peptide synthesis vessel. The mixture was agitated for 6 hours (for the last hour warmed to 30° C. with a water bath). The resin was filtered and the solids washed with 2 mL of TFA. The product containing filtrate and wash were combined. Wash with 420 mL of methyl-t-butyl ether (MTBE) that was cooled in an ice water bath. The solid product was collected by filtration. The solids were washed with 5 mL of MTBE. The solids were dried under vacuum to provide 1.19 g crude Compound of the Formula I.

### Purification Procedure 1

The resulting powder was dissolved in a small amount of DMSO and loaded onto a C18 column. The desired product was purified by reverse phase chromatography (0-55% acetonitrile in 50 mM ammonium carbonate buffer at pH 7.0). Acetonitrile was evaporated under reduced pressure, and the remaining aqueous buffer solution was frozen and removed by lyophilization. 256 mg (49.2%) of desired product was collected as a white powder.

### Purification Procedure 2

The compound of the Formula I was purified using a Biotage 60 g C18 ULTRA column with mobile phase A=0.1% TFA in water and mobile phase B=acetonitrile (ACN) (flow rate=45 mL/min, 1CV=90 mL, collection wavelength at 275 nm). The crude PSMA-617 in 20 mL of 0.1% TFA in water with 3 mL of ACN is loaded onto the column. The purification was run with 1CV 15% B, 7CV 15% B to 25% B, hold at 25% B. The product peak began to elute at approximately 5 CV, and fractions were collected. The fractions were examined using UPLC-MS, and the acceptable product fractions (≥98% at 275 nm) were combined, and the product solution was concentrated using a rotary evaporator to remove the ACN (final volume=59 mL). The compound of the Formula I was de-salted using a Biotage 30 g C18 ULTRA column is set up with mobile phase A=0.1% AcOH in water and mobile phase B=ACN (flow rate=25 mL/min, 1CV=45 mL, collection wavelength at 275 nm). To the aqueous PSMA-617 (59 mL) were charged 0.28 g ammonium acetate, 0.36 mL AcOH, and 3 mL ACN. The PSMA-617 solution was loaded onto the column, and the column was washed with 5 CV 5% B. The product was eluted from the column using 3CV 39% B. The product peak eluted at 6 CV and 1 fraction was collected. The product solution was concentrated using a rotary evaporator to remove ACN. The solids were isolated by lyophilization to provide 0.675 g of the Compound of the Formula I.

### Example 3: Characterization of PSMA-617 by Nuclear Magnetic Resonance and High Resolution MS ES+ (TOF)

¹H NMR (D₆-DMSO): 8.03 (t, 1H), 7.88 (t, 1H), 7.84 (d, 1H), 7.69-7.80 (m, 2H), 7.69 (s, 1H), 7.43-7.47 (m, 2H), 7.39 (dd, 1H), 6.29-6.33 (m, 2H), 4.50-4.52 (m, 1H), 4.08-4.11 (m, 1H), 3.99-4.02 (m, 1H), 3.46 (br s, 6H), 3.13 (br s, 2H), 3.10 (d, 1H), 2.89-3.04 (m, 17H), 2.68 (s, 4H), 2.22-2.27 (m, 2H), 2.07 (s, 1H), 1.91 (s, 1H), 1.57-1.74 (m, 5H), 1.46-1.50 (m, 2H), 1.31-1.35 (m, 3H), 1.22-1.26 (m, 3H), 1.06-1.12 (m, 1H), 0.80-0.82 (m, 2H).

Note: Six carboxylic acid protons are spread over the entire spectrum; additional resonances from residual acetic acid (1.91 ppm) and acetonitrile (2.07 ppm) are present.

¹³C NMR (D₆-DMSO): 175.09, 174.52, 174.14, 173.73, 170.99, 170.55, 170.11, 169.64, 157.26, 135.78, 132.86, 131.70, 127.81, 127.37, 127.32, 127.24, 127.22, 125.83, 125.24, 57.80, 55.34, 54.86, 53.85, 52.33, 51.78, 51.24, 50.52, 49.96, 49.84, 44.54, 43.77, 38.14, 37.86, 36.81, 31.65, 30.05, 29.61, 29.50, 28.63, 28.56, 28.38, 27.69, 22.41.

Note: Additional resonances from residual acetic acid (171.88 ppm, 20.98 ppm) and acetonitrile (1.068 ppm) are present.

High Resolution MS ES+ (TOF): M+H⁺=1042.5067 m/z (theoretical 1042.5096 m/z)

What is claimed is:

1. A process for preparing a compound of the formula 1 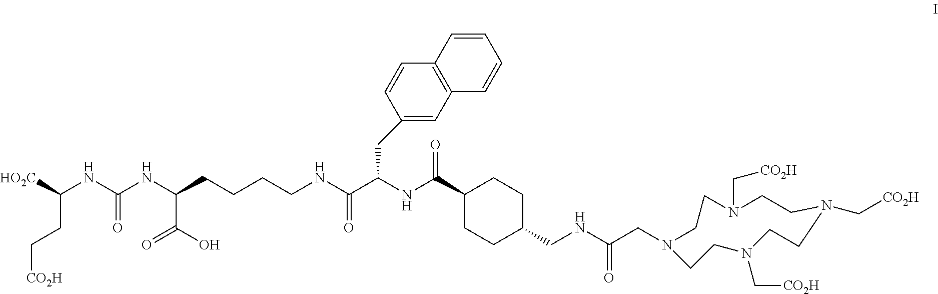
comprising either:
(i) contacting the resin-based compound of the formula 8 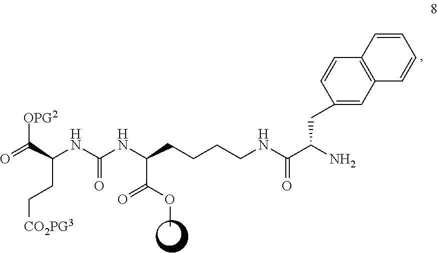
with a compound of the formula 9 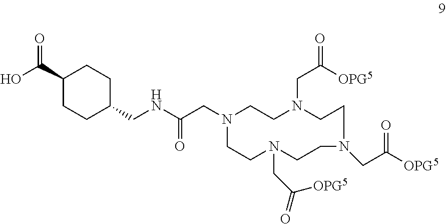
in the presence of a solvent, a coupling agent, and optionally a base and/or an additive to provide a resin-based compound of the formula 10 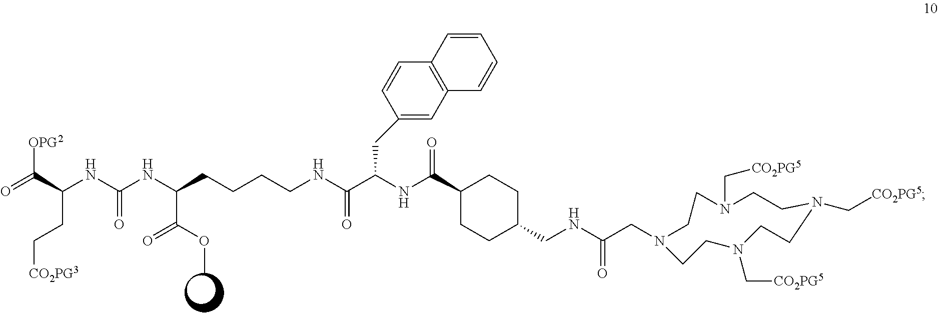
or
(ii) contacting the resin-based compound of the formula D′ 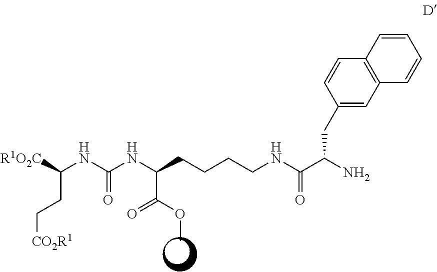
with PG¹-tranexamic acid in the presence of a polar aprotic solvent, a coupling agent, and a base to provide a resin-based compound of the formula E

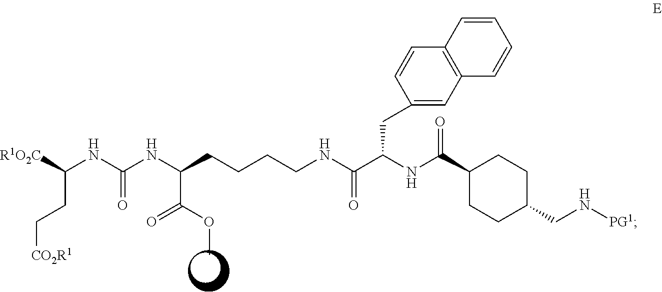

2. The process of claim 1, comprising one or more of
a. contacting a resin-based compound of the formula 1 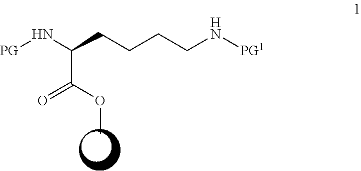
with a first deprotecting agent in the presence of a solvent to provide a resin-based compound of the formula 2 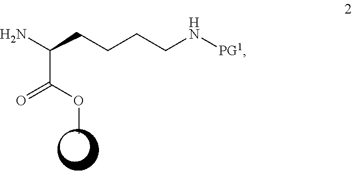
or
b. contacting the resin-based compound of the formula 2 with a compound of the formula 3 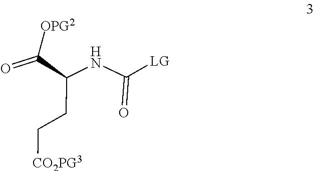
in the presence of an organic solvent and a base to provide a resin-based compound of the formula 4 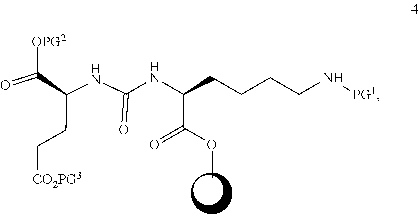
or
c. contacting the resin-based compound of the formula 4 with a second deprotecting agent in the presence of an organic solvent to provide a resin-based compound of the formula 5 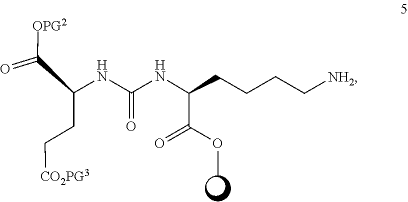
or
d. contacting the resin-based compound of the formula 5 with a compound of the formula 6 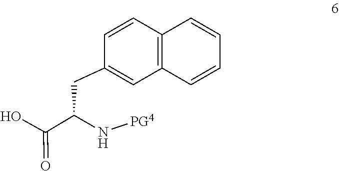
in the presence of a solvent, a coupling agent, and optionally a base and/or an additive to provide a resin-based compound of the formula 7 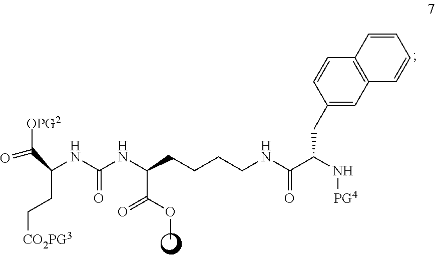
or
e. contacting the resin-based compound of the formula 7 with a third deprotecting agent in the presence of a solvent to provide a resin-based compound of the formula 8 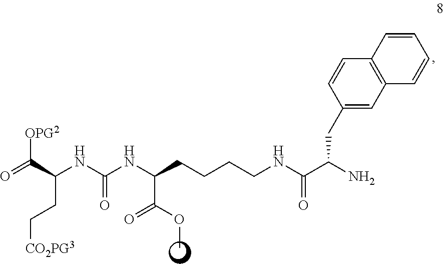
or
wherein PG, PG¹ and PG⁴ are amine protecting groups, PG², and PG³ are carboxyl protecting groups, and LG in compound 3 is a leaving group.

3. The process of claim 2, wherein PG, PG¹ and PG⁴ are selected from the group consisting of 9-fluorenylmethyl-carbonyl (Fmoc), benzyl carbamate (Cbz), t-butyloxy carbonyl (Boc), acetamide, trifluoroacetamide, p-toluenesulfonamide, triphemylmethyl, monomethoxytrityl (MMt), and 4-methyltrityl (Mtt).

4. The process of claim 2, wherein PG is 9-fluorenylmethyl-carbonyl.

5. The process of claim 2, wherein PG¹ is monomethoxytrityl (MMt) or 4-methyltrityl (Mtt).

6. The process of claim 2, wherein PG¹ is 4-methyltrityl (Mtt).

7. The process of claim 2, wherein PG⁴ is 9-fluorenylmethyl-carbonyl.

8. The process of claim 2, wherein PG², PG³, and PG⁵ are each t-butyl.

9. The process of claim 1, comprising (i).

10. The process of claim 9, further comprising:
contacting the resin-based compound of the formula 10 with at least one third deprotecting agent and/or a cleavage reagent in the presence of an organic solvent to provide the compound of the formula I.

11. The process of claim 1, comprising (ii).

12. The process of claim 11, further comprising:
a) contacting the resin-based compound of the formula E with the first deprotecting agent in a polar aprotic solvent to provide a resin-based compound of the formula E′ 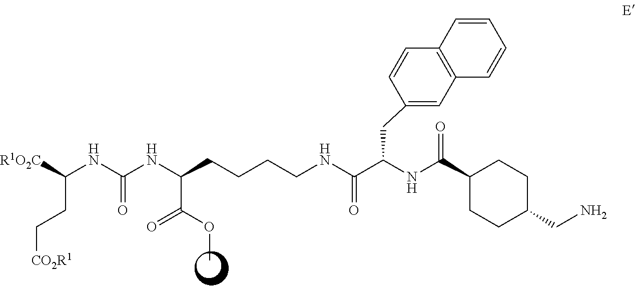 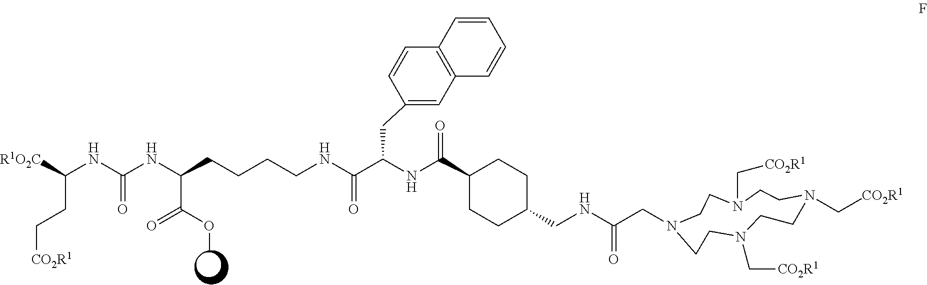
or
b) contacting the resin-based compound of the formula E′ with a compound of the formula E^{2′} 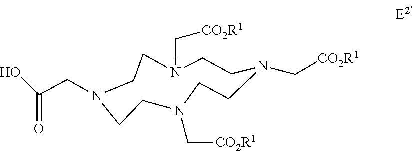
in the presence of a polar aprotic solvent, a coupling agent, and a base to provide a resin-based compound of the formula F 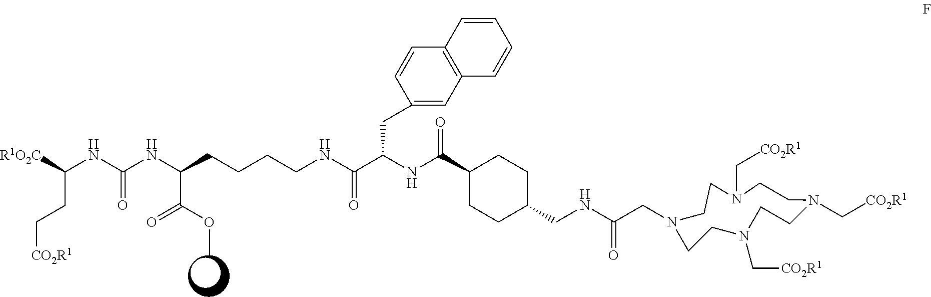
or
c) contacting the resin-based compound of the formula F with a third deprotecting agent and/or a cleavage reagent in an organic solvent,
wherein PG¹ and PG² are amine protecting groups, and R¹ is a C₁-C₄ alkyl or a cyclohexyl.

13. A compound of the formula 9′ 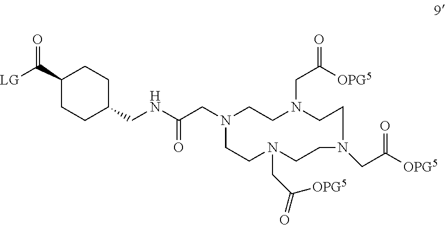
wherein LG is a leaving group and each PG⁵ is a carboxyl protecting group.

14. The compound of claim 13, wherein LG is selected from the group consisting of 1-(λ¹-oxidaneyl)-1H-benzo[d][1,2,3]triazole, 3-(λ¹-oxidaneyl)-3H-[1,2,3]triazolo[4,5-b]pyridine, ethyl cyano (hydroxyimino)acetate (Oxyma), bromide, 3-(λ¹-oxidaneyl)benzo[d][1,2,3]triazin-4 (3H)-one, 6-chloro-1-(λ¹-oxidaneyl)-1H-benzo[d][1,2,3]triazole, 2,4-dimethoxy-6-(λ¹-oxidaneyl)-1,3,5-triazine, etabonic acid, propylphosphonic acid, dicyclohexylurea, 1-(3-(dimethyl-λ⁴-azaneyl) propyl)-3-ethylurea, and imidazolyl.

15. The compound of claim 14, wherein each PG⁵ is t-butyl.

16. A compound of the formula 9 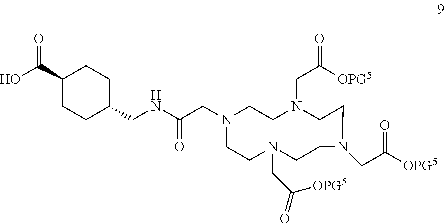
wherein each PG⁵ is a carboxyl protecting group.

17. The compound of claim 16, wherein each PG⁵ is t-butyl.
